(19) United States
(12) Patent Application Publication
(10) Pub. No.: US 2020/0172530 A1
(43) Pub. Date: Jun. 4, 2020

(54) Small Molecule Sensitization of BAX Activation for Induction of Cell Death

(71) Applicant: Dana-Farber Cancer Institute, Inc., Boston, MA (US)

(72) Inventors: Loren D. Walensky, Newton, MA (US); Jonathan Pritz, Coral Springs, FL (US); Franziska Wachter, Boston, MA (US)

(21) Appl. No.: 16/492,841

(22) PCT Filed: Mar. 14, 2018
(86) PCT No.: PCT/US2018/022345

§ 371 Date: Sep. 10, 2019

Related U.S. Application Data
(60) Provisional application No. 62/471,174, filed on Mar. 14, 2017.

(51) Int. Cl. C07D 417/12; C07D 209/08; C07D 207/34; C07D 285/18; C07D 295/26; C07D 307/68; C07D 333/72; C07K 14/47

(52) U.S. Cl. CPC C07D 417/12; C07D 209/08; C07D 207/34; C07D 285/18; A61K 45/06; C07D 307/68; C07D 333/72; C07K 14/47; C07D 295/26

(57) ABSTRACT

Provided herein are compositions comprising a compound of Formulas I, II, or III, and compositions a compound comprising a moiety of Formula IV, which are useful for sensitizing and/or activating pro-apoptotic activity of BAX. Methods of treating diseases associated with BAX (e.g., cancer) and methods of identifying compounds which sensitize and/or activate the pro-apoptotic activity of a BAX polypeptide are also provided.

### FEDERALLY SPONSORED RESEARCH OR DEVELOPMENT

This invention was made with Government support under Grant Nos. NIH1R35, CA197583, awarded by the National Institutes of Health. The Government has certain rights in the invention.

### TECHNICAL FIELD

The present application provides compositions containing a compound of Formulas I, II, or III, and compositions containing a compound comprising a moiety of Formula IV, which are useful for sensitizing and/or activating pro-apoptotic activity of BAX. Methods of treating diseases associated with BAX (e.g., cancer) and methods of identifying compounds which sensitize and/or activate the pro-apoptotic activity of a BAX polypeptide are also described.

### BACKGROUND

BAX is a 21 kDa globular protein composed of nine α-helices and functions as a critical effector of the BCL-2 family-regulated mitochondrial apoptotic pathway. An α5/α6 hairpin forms the protein's hydrophobic core, the juxtaposition of α-helices 1 and 6 creates a ligand-interaction surface that regulates the initiation of BAX activation, and at the opposite face of the protein the auto-inhibitory α9 helix resides in a hydrophobic groove composed of portions of α-helices 2, 3 and 4 (see e.g., Suzuki et al, Cell, 200, 103:645-654). BAX is an apoptotic regulator that can be transformed from a cytosolic monomer into a lethal mitochondrial oligomer.

### SUMMARY

The present application provides, inter alia, a composition, comprising a compound of Formula I:

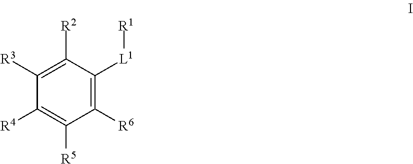

or a pharmaceutically acceptable salt thereof, and one or more pharmaceutically acceptable carriers, wherein: L¹ is selected from the group consisting of a bond, C₁₋₃ alkylene, —O—, —O(C₁₋₃ alkylene)-, C₁₋₃ cyanoalkylene, —S—, —SO₂—, —S(C₁₋₃ alkylene)-, and —C(O)—; R¹ is selected from the group consisting of halo, OH, C₁₋₃ alkyl, C₁₋₃ haloalkyl, NH₂, CN, phenyl, 5-6 membered heteroaryl, and 5-6 membered heterocycloalkyl, wherein the phenyl, 5-6 membered heteroaryl, and 5-6 membered heterocycloalkyl are each optionally substituted by 1, 2, or 3 independently selected R^{A} groups; R² is selected from the group consisting of H, halo, OH, CN, C₁₋₃ alkyl, and C(O)OC₁₋₃ alkyl; R³ is selected from the group consisting of H, halo, OH, NH₂, C(O)C₁₋₃ alkyl, and C(S)C₁₋₃ alkyl; R⁴ is selected from the group consisting of H, halo, OH, NH₂, CN, C₁₋₃ alkyl, C₁₋₃ haloalkyl, and O(C₁₋₃ cyanoalkyl); R⁵ is selected from the group consisting of H, halo, OH, NH₂, and C(O)C₁₋₃ alkyl; R⁶ is selected from the group consisting of H, halo, OH, CN, C₁₋₃ alkyl, and C(O)OC₁₋₃ alkyl; and each R^{A} is independently selected from the group consisting of OH, NH₂, CN, C₁₋₃ alkyl, C₁₋₃ hydroxyalkyl, C(O)OH, C(O)C₁₋₃ alkyl, and C(O)N(C₁₋₃ alkyl)₂, wherein the C₁₋₃ alkyl group is optionally substituted by NH₂.

In some embodiments, L¹ of Formula I is selected from the group consisting of a bond, —CH₂—, —O—, —OCH₂—, —CH(CN)—, —S—, —SO₂—, —SCH₂—, and —C(O)—. In some embodiments, L¹ is —O—, —CH₂—, or —OCH₂—.

In some embodiments, R¹ of Formula I is selected from the group consisting of Cl, CH₃, CF₃, NH₂, CN, phenyl, 5-6 membered heteroaryl, and 5-6 membered heterocycloalkyl, wherein the phenyl, 5-6 membered heteroaryl, and 5-6 membered heterocycloalkyl are each optionally substituted by 1 or 2 independently selected R^{A} groups.

In some embodiments, R¹ of Formula I is selected from the group consisting of Cl, CH₃, CF₃, NH₂, CN, phenyl, pyridyl, furanyl, thienyl, pyrrolyl, thiazolyl, oxazolyl, pyrazolyl, 1,2,4-thiadiazolyl, piperidinyl, morpholinyl, and 4,5-dihydrothiazolyl wherein the phenyl, pyridyl, furanyl, thienyl, pyrrolyl, thiazolyl, oxazolyl, pyrazolyl, 1,2,4-thiadiazolyl, piperidinyl, morpholinyl, and 4,5-dihydrothiazolyl are each optionally substituted by 1 or 2 independently selected R^{A} groups.

In some embodiments, each R^{A} of Formula I is independently selected from the group consisting of OH, NH₂, CN, CH₃, CH₂OH, CH₂CH₂NH₂, C(O)OH, C(O)CH₃, and C(O)N(CH₃)₂.

In some embodiments, R¹ of Formula I is phenyl which is optionally substituted by 1 or 2 independently selected R^{A} groups. In some embodiments, R¹ of Formula I is phenyl, 4-hydroxyphenyl, 3-hydroxyphenyl, 4-aminophenyl, 4-carboxylphenyl, or 4-hydroxymethylphenyl.

In some embodiments, R² of Formula I is selected from the group consisting of H, Cl, CN, CH₃, and C(O)OCH₃. In some embodiments, R² of Formula I is H or CH₃.

In some embodiments, R³ of Formula I is selected from the group consisting of H, F, Cl, NH₂, C(O)CH₃, and C(S)CH₃. In some embodiments, R³ of Formula I is H.

In some embodiments, R⁴ of Formula I is selected from the group consisting of H, Cl, NH₂, CN, CH₃, CF₃, and OCH₃CN. In some embodiments, R⁴ of Formula I is H or OH.

In some embodiments, R⁵ of Formula I is selected from the group consisting of H, F, Cl, NH₂, and C(O)CH₃. In some embodiments, R⁵ of Formula I is H or NH₂.

In some embodiments, R⁶ of Formula I is selected from the group consisting of H, Cl, CN, CH₃, and C(O)OCH₃. In some embodiments, R⁶ of Formula I is H.

In some embodiments, the compound of Formula I is selected from the group consisting of:

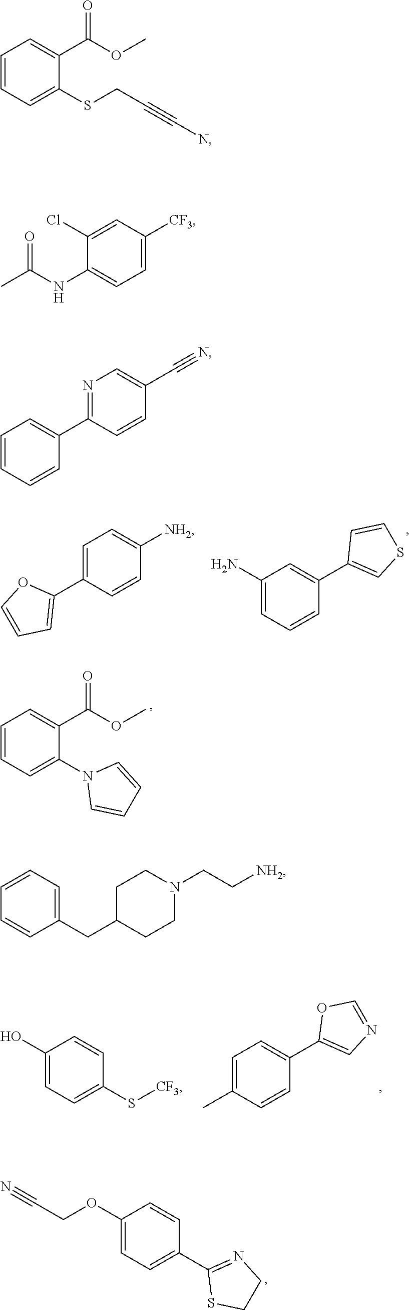

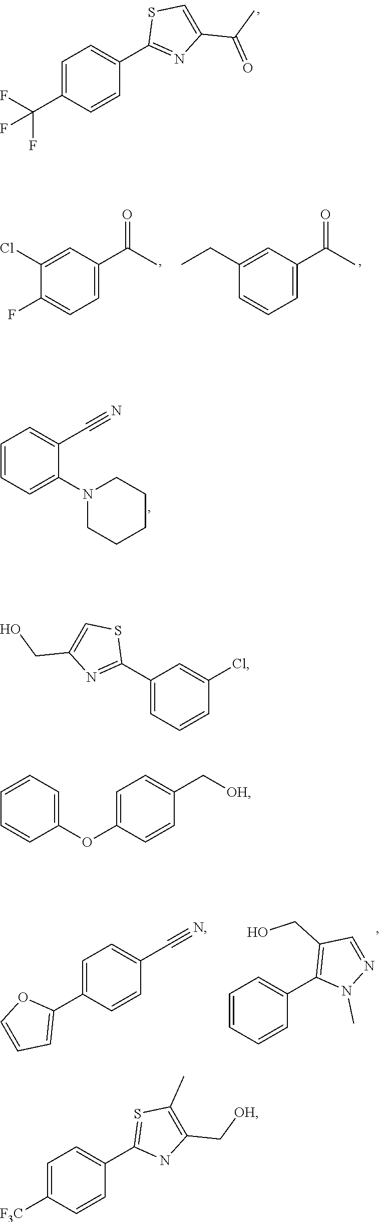

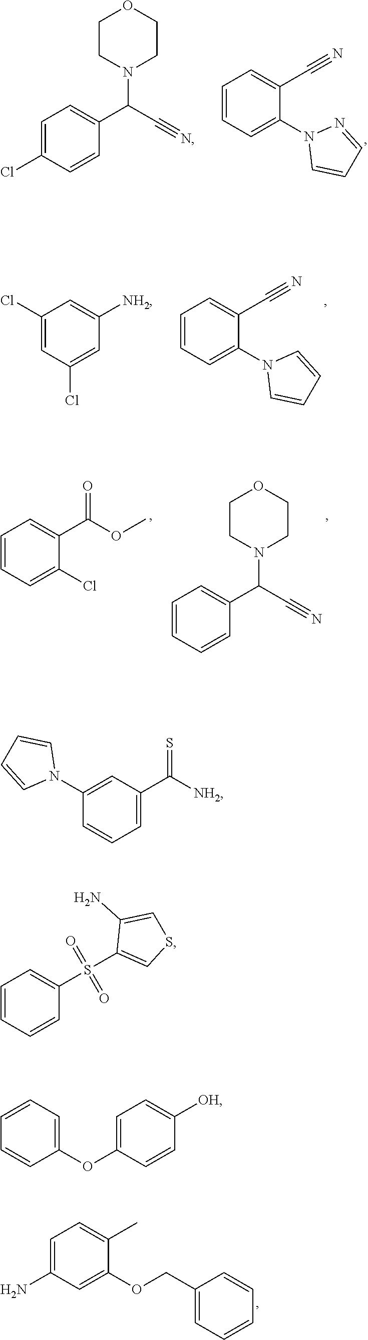

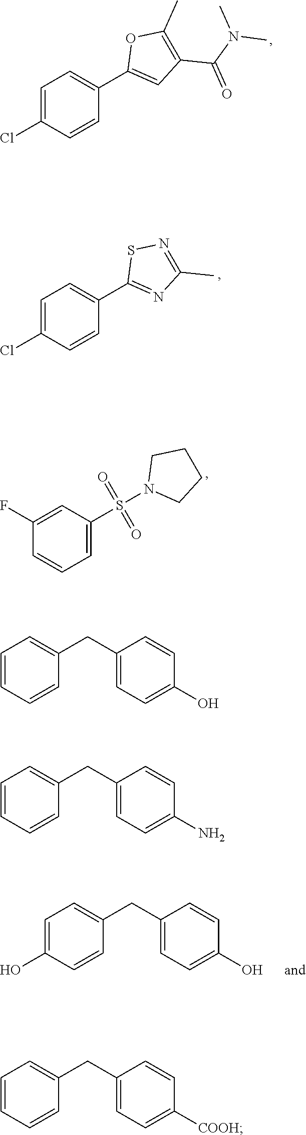

or a pharmaceutically acceptable salt thereof.

In some embodiments, the compound of Formula I is:

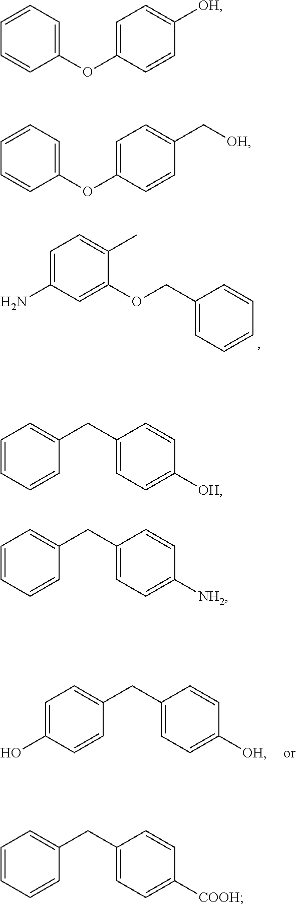

or a pharmaceutically acceptable salt thereof.

The present application further provides a composition, comprising a compound of Formula II:

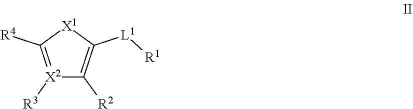

or a pharmaceutically acceptable salt thereof, and one or more pharmaceutically acceptable carriers, wherein: X¹ is NH or S; X² is C or N; L¹ is selected from the group consisting of a bond, —C(O)—, —C(O)O—, and —SO₂—; R¹ is selected from the group consisting of C₁₋₃ alkyl, NH₂, di(C₁₋₃ alkyl)amino, and a 5-6 membered heterocycloalkyl; R² is selected from the group consisting of H, halo, C₁₋₃ alkyl, and C(O)OC₁₋₃ alkyl; R³ is selected from the group consisting of H, C₁₋₃ alkyl, and 5-6 membered heteroaryl; or R³ is absent when X² is N; and R⁴ is selected from the group consisting of H and C₁₋₃ alkyl.

In some embodiments, X¹ of Formula II is NH.

In some embodiments, X¹ of Formula II is S.

In some embodiments, X² of Formula II is C.

In some embodiments, X² of Formula II is N.

In some embodiments, R¹ of Formula II is selected from the group consisting of CH₃, CH₂CH₃, NH₂, N(CH₂CH₃)₂, piperidinyl, and dihydrothiophen-3(2H)-onyl.

In some embodiments, -L¹-R¹ of Formula II forms a group selected from the group consisting of NH₂, C(O)OCH₃, C(O)OCH₂CH₃, C(O)N(CH₂CH₃)₂, SO₀₂-piperidinyl, and dihydrothiophen-3(2H)-onyl.

In some embodiments, R² of Formula II is selected from the group consisting of H, Cl, CH₃, and C(O)OCH₂CH₃.

In some embodiments, R³ of Formula II is selected from the group consisting of H, CH₃, CH₂CH₃, and thienyl.

In some embodiments, R⁴ of Formula II is selected from the group consisting of H and C₁₋₃ alkyl.

In some embodiments, the compound of Formula II is selected from the group consisting of:

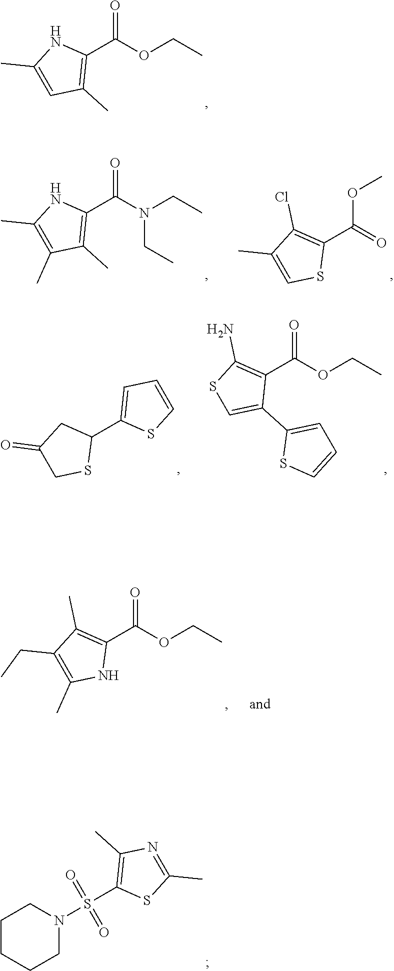

or a pharmaceutically acceptable salt thereof.

The present application further provides a composition, comprising a compound of Formula III:

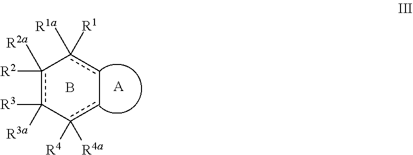

or a pharmaceutically acceptable salt thereof, and one or more pharmaceutically acceptable carriers, wherein: [symbol] refers to a single bond or a double bond; Ring A forms a fused ring with Ring B and Ring A is selected from the group consisting of a 5-6 membered cycloalkyl, a 5-6 membered heteroaryl, and a 5-6 membered heterocycloalkyl, wherein Ring A is optionally substituted by 1, 2, or 3 independently selected R^{A} groups; R¹ is selected from the group consisting of H, C(O)OC₁₋₃ alkyl, OC(O)C₁₋₃ alkyl, C(S)NH₂, and ═N—OH; R^{1a} is H; or R^{1a} is absent when the carbon atom to which R^{1a} is attached forms a double bond; R² is selected from the group consisting of H and halo; R^{2a} is H; or R^{2a} is absent when the carbon atom to which R^{2a} is attached forms a double bond; R³ is selected from the group consisting of H, halo, C₁₋₃ alkyl, C₁₋₃ hydroxyalkyl, NHC(O)C₁₋₃ alkyl, and (C₁₋₃ alkylene)NHC₁₋₃ alkyl; R^{3a} is C₁₋₃ alkyl; or R^{3a} is absent when the carbon atom to which R^{3a} is attached forms a double bond; R⁴ is selected from the group consisting of H and C₁₋₃ alkyl; R^{4a} is H; or R^{4a} is absent when the carbon atom to which R^{4a} is attached forms a double bond; and each R^{A} is independently selected from the group consisting of ═O, ═S, CN, C₁₋₃ alkyl, C₁₋₃ hydroxyalkyl, S(C₁₋₃ alkyl), and C(O)OH.

In some embodiments, Ring A is a 5-6 membered heteroaryl which is optionally substituted by 1, 2, or 3 independently selected R^{A} groups. In some embodiments, Ring A is a 5-6 membered heterocycloalkyl groups which is optionally substituted by 1, 2, or 3 independently selected R^{A} groups. In some embodiments, each R^{A} of Formula III is independently selected from the group consisting of ═O, ═S, CN, CH₃, CH₂OH, SCH₃, and C(O)OH. In some embodiments, Ring A is an unsubstituted 5-6 membered cycloalkyl.

In some embodiments, Ring A is selected from the group consisting of:

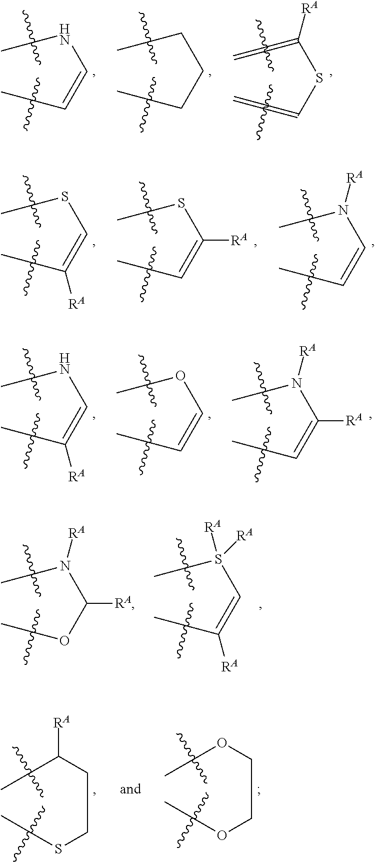

wherein each [symbol] indicates the bonds connecting the fused Ring A and Ring B.

In some embodiments, R¹ of Formula III is selected from the group consisting of H, C(O)OCH₃, OC(O)CH₃, C(S)NH₂, and ═N—OH.

In some embodiments, R² of Formula III is selected from the group consisting of H and Cl.

In some embodiments, R^{2a} of Formula III is H. In some embodiments, R^{2a} of Formula III is absent.

In some embodiments, R³ of Formula III is selected from the group consisting of H, Cl, CH₃, CH₂OH, NHC(O)CH₃, and CH₂NHCH₃.

In some embodiments, R^{3a} of Formula III is CH₃. In some embodiments, R^{3a} of Formula III is absent.

In some embodiments, R⁴ of Formula III is selected from the group consisting of H and CH₃.

In some embodiments, the compound of Formula III is selected from the group consisting of:

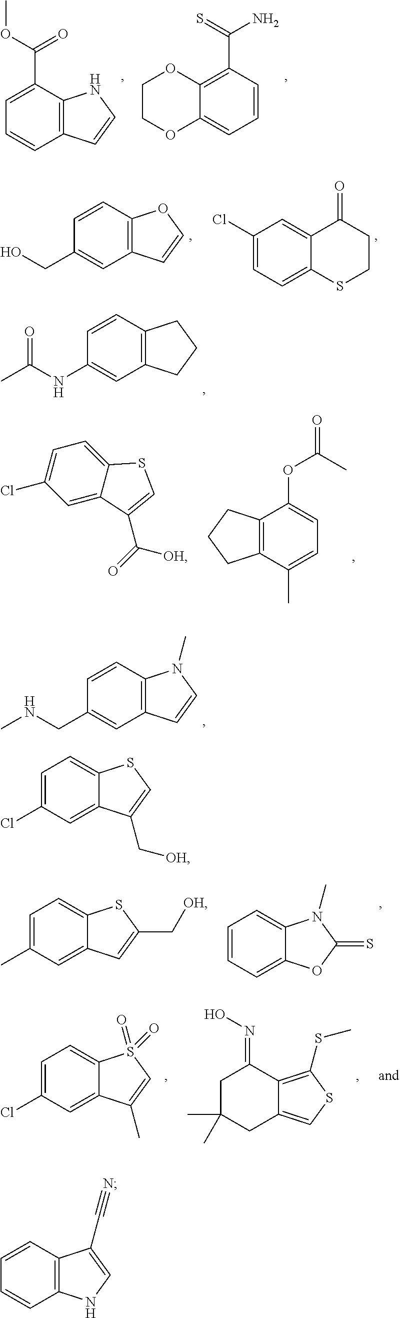

or a pharmaceutically acceptable salt thereof.

The present application further provides a composition, comprising a compound comprising a moiety of Formula IV:

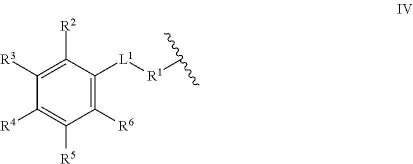

or a pharmaceutically acceptable salt thereof, and one or more pharmaceutically acceptable carriers, wherein: L¹ is selected from the group consisting of a bond, C₁₋₃ alkylene, —O—, —O(C₁₋₃ alkylene)-, C₁₋₃ cyanoalkylene, —S—, —SO₂—, —S(C₁₋₃ alkylene)-, and —C(O)—; R¹ is selected from the group consisting of phenylene, 5-6 membered heteroarylene, and 5-6 membered heterocycloalkylene, each of which is optionally substituted by 1, 2, or 3 independently selected R^{A} groups; R² is selected from the group consisting of H, halo, OH, CN, C₁₋₃ alkyl, and C(O)OC₁₋₃ alkyl; R³ is selected from the group consisting of H, halo, OH, NH₂, C(O)C₁₋₃ alkyl, and C(S)C₁₋₃ alkyl; R⁴ is selected from the group consisting of H, halo, OH, NH₂, CN, C₁₋₃ alkyl, C₁₋₃ haloalkyl, and O(C₁₋₃ cyanoalkyl); R⁵ is selected from the group consisting of H, halo, OH, NH₂, and C(O)C₁₋₃ alkyl; R⁶ is selected from the group consisting of H, halo, OH, CN, C₁₋₃ alkyl, and C(O)OC₁₋₃ alkyl; and each R^{A} is independently selected from the group consisting of OH, NH₂, CN, C₁₋₃ alkyl, C₁₋₃ hydroxyalkyl, C(O)OH, C(O)C₁₋₃ alkyl, and C(O)N(C₁₋₃ alkyl)₂, wherein the C₁₋₃ alkyl group is optionally substituted by NH₂.

In some embodiments, L¹ of Formula IV is selected from the group consisting of a bond, —CH₂—, —O—, —OCH₂—, —CH(CN)—, —S—, —SO₂—, —SCH₂—, and —C(O)—.

In some embodiments, R¹ of Formula IV is phenylene optionally substituted by 1 or 2 independently selected R^{A} groups.

In some embodiments, each R^{A} of Formula IV is independently selected from the group consisting of OH, NH₂, CN, CH₃, CH₂OH, CH₂CH₂NH₂, C(O)OH, C(O)CH₃, and C(O)N(CH₃)₂.

In some embodiments, R² of Formula IV is selected from the group consisting of H, Cl, CN, CH₃, and C(O)OCH₃.

In some embodiments, R³ of Formula IV is selected from the group consisting of H, F, Cl, NH₂, C(O)CH₃, and C(S)CH₃.

In some embodiments, R⁴ of Formula IV is selected from the group consisting of H, Cl, NH₂, CN, CH₃, CF₃, and OCH₃CN.

In some embodiments, R⁵ of Formula IV is selected from the group consisting of H, F, Cl, NH₂, and C(O)CH₃.

In some embodiments, R⁶ of Formula IV is selected from the group consisting of H, Cl, CN, CH₃, and C(O)OCH₃.

The present application further provides a method of sensitizing and/or activating the pro-apoptotic activity of BAX, comprising contacting a cell sample or tissue sample comprising BAX with a composition provided herein.

The present application further provides a method of sensitizing and/or activating pro-apoptotic activity of BAX in a subject, comprising administering to the subject a composition provided herein.

The present application further provides a method of treating cancer in a subject, comprising administering to the subject a therapeutically effective amount of a composition provided herein.

In some embodiments, the cancer is selected from the group consisting of breast cancer, prostate cancer, lymphoma, skin cancer, pancreatic cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma. In some embodiments, the cancer is leukemia. In some embodiments, the leukemia is selected from the group consisting of acute lymphocytic leukemia, chronic lymphocytic leukemia, chronic granulocytic leukemia, acute granulocytic leukemia, acute myelogenous leukemia, chronic myelogenous leukemia, and hairy cell leukemia. In some embodiments, the leukemia is selected from the group consisting of acute lymphoblastic leukemia, acute myelogenous leukemia, chronic lymphoblastic leukemia, and chronic myelogenous leukemia.

The present application further provides a method for identifying a compound which sensitizes and/or activates the pro-apoptotic activity of a BAX polypeptide, the method comprising:

a) contacting a binding site of said BAX polypeptide comprising an amino acid sequence of SEQ ID NO:1 with a compound in vitro under conditions suitable for sensitizing and/or activating the pro-apoptotic activity of the BAX polypeptide; and

b) determining whether the compound binds to one or more amino acid residues selected from the group consisting of Ile80, Ala81, Ala82, Val83, Asp84, Thr85, Asp86, Ser87, Pro88, Val91, Phe116, Lys119, Leu120, Val121, Lys123, Ala124, Thr127, Leu132, and Ile136;

wherein the binding site of the BAX polypeptide comprises the junction of the α3-α4 and α5-α6 hairpins of the BAX polypeptide.

In some embodiments, the determining step is performed by saturation transfer difference NMR, HSQC NMR, surface plasmon resonance, biolayer interferometry, or competitive fluorescence polarization assay.

In some embodiments, binding of the compound to the BAX polypeptide causes a change in the signal of the NMR spectrum of the compound.

In some embodiments, the method further comprising detecting activation of the BAX polypeptide by the compound. In some embodiments, the detecting step comprises performing an assay selected from the group consisting of detecting BAX oligomerization, antibody-based detection of BAX conformers, a mitochondrial cytochrome c release assay, a liposomal release assay, a cell death assay, a mitochondrial or cellular morphology assay, a mitochondrial calcium flux assay, a mitochondrial transmembrane quantitation assay, and quantitation of caspase 3 activity or annexin V binding.

In some embodiments, the compound binds to said binding site with an affinity of <1 mM. In some embodiments, the compound sensitizes activation of the pro-apoptotic activity of the BAX polypeptide. In some embodiments, the compound activates the pro-apoptotic activity of the BAX polypeptide.

In some embodiments, the method further comprises administration of an additional therapeutic agent which activates pro-apoptotic activity of BAX. In some embodiments, the additional therapeutic agent is BIM SAHB_{A2}.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### DESCRIPTION OF DRAWINGS

FIG. 1a shows BAX, which contains a series of surface grooves that regulate its pro-apoptotic activity, including BH3-binding trigger and canonical sites, and inhibitory BCL-2 BH4 and vMIA interaction pockets.

FIG. 1b shows identification of compounds (also referred to as BAX-interacting fragments (BIFs)) as described herein by sequential STD-NMR screening in pools of 10, 3, and then singlet, yielding 56 candidate BIFs.

FIG. 1c shows that BIF-44 has no independent effect on the liposomes (black, left), minimal direct BAX activation activity (black, middle), but notably enhances the kinetics and quantity of liposomal release upon addition of BIM SAHB_{A2} (black, right), exceeding the maximal level of release achieved by the BIM SAHB_{A2} and BAX combination alone (grey, right). Error bars are mean±SD for experiments performed in technical triplicate, and repeated at least twice with similar results using independent liposomal and protein preparations.

FIG. 1d shows that competitive STD NMR demonstrates that the BIF-44 STD signal is unaffected by co-incubation with BIM SAHB_{A2}.

FIGS. 2a-2b show that liposomal release assays demonstrate little to no direct, BAX-activating effect of BIF-44 across a broad dose range, but sensitizes BH3-triggered direct BAX activation upon co-incubation with BIM SAHB_{A2} (FIG. 2b). Error bars are mean±SD for experiments performed in technical triplicate.

FIGS. 2c-2d show that competitive fluorescence polarization assays (FPA) demonstrate that BIF-44 does not effectively compete with FITC BIM SAHB_{A2} for BAX interaction (FIG. 2c), yet does compete with FITC-vMIA in dose responsive fashion (FIG. 2d). The corresponding N-terminal acetylated peptides serve as positive controls for competition in each assay: Ac-BIM SAHB_{A2}, blue (FIG. 2c); AcvMIA, purple (FIG. 2d). Error bars are mean±SD for experiments performed in technical quadruplicate.

FIG. 2e shows that competitive STD-NMR demonstrate suppression of the BIF-44 STD signal (black) upon co-incubation with vMIA peptide (grey), but not BIM SAHB_{A2}, consistent with the competitive FPA results shown in FIGS. 2c-2d. Data are representative of at least two independent experiments.

FIGS. 3a-3e show structure-activity relationships of BIF-44 analogs. Chemical structures (left), STD binding (grey) and BAX-mediated liposomal release sensitization activity of BIF-44 analogs are provided. Error bars are mean±SD for liposomal release experiments performed in technical triplicate (right). Data are representative of at least two independent experiments.

FIGS. 4a-4e show that BIF-44 targets the vMIA-binding region of BAX and influences conformational dynamics.

FIG. 4a shows measured chemical shift changes of ¹⁵N-BAX upon addition of BIF-44 (6:1, BIF:BAX), plotted as a function of BAX residue number. The most prominent effects, reflecting chemical shift changes above the 2 SD cutoff (≥0.018 ppm significance threshold), are colored red and localize to the junction of the α3-α4, and α5-α6 hairpins. Significant changes at the 1 SD cutoff threshold (≥0.012 ppm significance threshold), are colored orange and encompass internal residues of the α5 and α6 core and discrete, juxtaposed residues of α1 and α2.

FIG. 4b shows residues that are represented as red and orange bars in the residue plot of FIG. 4a are mapped accordingly onto the ribbon diagrams of monomeric BAX (PDB ID: 1F16). The most prominent chemical shift changes (2 SD cutoff) localize to the region implicated in the vMIA peptide (purple) interaction. A second cluster of chemical shift changes (1 SD cutoff) localize to internal and juxtaposed residues of α5, α6 and α1, α2, suggestive of allosteric sensing from the adjacent hydrophobic core to the α1-loop-α2 region of the BAX N-terminal face.

FIG. 4c shows molecular docking of BIF-44 based on the observed chemical shift changes of ¹⁵N-BAX (black, 2 SD, grey 1 SD) upon BIF-44 titration. BIF-44 is shown engaging a deep cleft formed by the hydrophobic α5 and α6 helices, and the α3-α4 hairpin of BAX on the surface (left) and ribbon (middle, right) views.

FIG. 4d shows RMSF values for the Ca of each BAX residue over the course of the 100 ns molecular dynamics simulation for BAX in the presence (grey) or absence (black) of BIF-44.

FIG. 4e shows the difference in RMSF (ARMSF) between the unliganded and liganded forms of BAX. Residues above one SD threshold are shown in grey, indicate increased mobility upon BIF-44 binding, and localized to the α1-α2 region of BAX. Residues from the unstructured portions at the N- and C-termini (residues 1-15 and 188-192, respectively) are excluded from the plot.

FIGS. 5a-5d show HXMS reveals allosteric deprotection of the α1-α2 loop and BAX BH3 domain upon BIF-44 binding.

FIG. 5a shows that the addition of BIF-44 to BAX (30 μM, 10:1 BIF:BAX) in a liposomal environment triggers a regiospecific increase in deuterium incorporation compared to unliganded BAX, as measured by HXMS. The relative difference plot reflects the relative deuterium incorporation of BIF-44/BAX minus the relative deuterium incorporation of BAX. Dark gray shading represents changes in the plot that are below the significance threshold of 0.5 Da, whereas light gray shading and the white region highlight changes above the baseline significance threshold of 0.5 Da and the more stringent threshold of 0.8 Da, respectively. Data are representative of at least two independent experiments.

FIG. 5b shows that the region of BIF-44-induced deprotection encompasses peptide fragments corresponding to amino acids 46-74, which are highlighted in black on the ribbon diagram (left, PDB ID: 1F16) and amino acid sequence (SEQ ID NO:1, right), and map to the critical α1-α2 loop and BH3 regions of BAX.

FIGS. 5c-5d show that the deprotection induced by BIF-44 is suppressed by co-incubation with an anti-BAX BH3 antibody (FIG. 5c), but not the BAX 6A7 antibody (FIG. 5d), which binds to N-terminal residues of conformationally-activated BAX. The BAX amino acid sequences recognized by the BAX BH3 and 6A7 antibodies are underlined in light grey and dark grey, respectively. The relative difference plots reflect the relative deuterium incorporation of BIF-44/BAX/BH3 Ab (FIG. 5c) and BIF-44/BAX/6A7 Ab (FIG. 5d) minus the relative deuterium incorporation of BAX. Data are representative of at least two independent experiments.

FIGS. 6a-6c show BIF-44 sensitized the BH3-triggered conformational activation and cytochrome c release activity of BAX.

FIG. 6a shows comparative HXMS profiles of BAX in the presence of liposomes upon exposure to BIF-44 (light grey border), BIM SAHB_{A2} (dark grey), or both ligands (black). The relative difference plots reflect the relative deuterium incorporation of BIF-44/BAX (light grey border), BIM SAHB_{A2}/BAX (dark grey), and BIF-44/BIM SAHB_{A2}/BAX (black) minus the relative deuterium incorporation of BAX. Dark gray shading represents changes in the plot that are below the significance threshold of 0.5 Da, whereas light gray shading and the white region highlight changes above the baseline significance threshold of 0.5 Da and the more stringent threshold of 0.8 Da, respectively. Data are representative of at least two independent experiments.

FIG. 6b shows that the prominent region of deprotection (α1, α1-α2 loop, and α2) induced by treating BAX with the synergistic BIF-44/BIM SAHB_{A2} combination is highlighted in green on the ribbon diagram (PDB ID: 1F16) and amino acid sequence (SEQ ID NO:1).

FIG. 6c shows BIF-44 dose-responsively sensitized BIM SAHB_{A2}-triggered, BAX-mediated cytochrome c release from isolated Alb^{Cre}Bax^{f/f}Bak^{-/-} mouse liver mitochondria. Error bars are mean±SD for experiments performed in technical triplicate, and repeated twice more with similar results using independent preparations and treatments of mitochondria.

FIGS. 7a-7c show STD and CPMG NMR analysis of the BIF-44/BAX interaction.

FIGS. 7a-7b show STD NMR of BIF-44 in the presence and absence of BAX protein. The off-resonance condition shows no effect on the aromatic region of BIF-44 in the presence or absence of BAX (FIG. 7a). An STD signal (STD=off resonance minus on resonance) for BIF-44 is only detected in the presence of BAX, reflective of ligand-protein interaction (FIG. 7b).

FIG. 7c shows CPMG NMR of BIF-44 in the presence and absence of BAX. The addition of BAX protein enhanced the transverse relaxation rate, R2, of the BIF-44 ligand, which is reflected by a sharp decrease in 1H-NMR signal and indicative of ligand-protein interaction.

FIGS. 8a-8b show FITC-BIM SAHB_{A2} and vMIA peptides directly bind to BAX. Fluorescence polarization assays demonstrate direct interaction between BAX and the FITC-BIM SAHB_{A2} (FIG. 8a) and FITC-vMIA (FIG. 8b) peptides. Error bars are mean±SD for experiments performed in quadruplicate.

FIG. 9 shows ¹⁵N-HSQC analysis of BAX upon BIF-44 titration. Measured chemical shift changes of ¹⁵N-BAX upon addition of BIF-44 at ratios of 4:1, 6:1, and 8:1 (BIF:BAX), plotted as a function of BAX residue number. Significant changes at a 1 SD cutoff threshold for each dosing ratio (≥0.012, 0.018, and 0.022 ppm significance thresholds) are colored black, blue, and red, respectively.

FIG. 10 shows isothermal titration calorimetry (ITC) measurements which demonstrates that BIF-44 binds to BAX with a dissociation constant (KD) of 37±12 M. Raw heats of binding were fitted to single site binding model. BIF-44 at a concentration of 1 mM was injected into the cell (2 μL per injection) containing 0.15 mM BAX. Samples were diluted to the indicated concentrations in 20 mM potassium phosphate buffer, pH 6.2, 1% DMSO.

FIG. 11 shows that BIF-44 sensitization of BAX-mediated liposome release is independent of the order of addition. The same level of BAX activation is achieved whether BIF-44 is added simultaneously (left), before (right), or after (middle) the addition of BIM SAHB_{A2}. The concentration of BAX and BIF-44 was 750 nM and 113 μM (150×), respectively. Samples were diluted in liposome release assay buffer (10 mM HEPES, 200 mM KCl, 1 mM MgCl₂, pH 7.0).

FIG. 12 shows that BIF-44 does not exhibit line broadening in the ¹H-NMR spectrum, unlike two known small molecule aggregators, 4-ADPA and I4PTH. Samples were diluted to the indicated concentrations in 20 mM potassium phosphate buffer, pH 6.2, 10% D₂O.

FIG. 13 shows that BIF-44 does not exhibit rapid or dose dependent decrease in T2 decay, while the aggregating compound 4-ADPA demonstrates rapid T2 decay (top, gray). BIF-44 had a long decay time that was independent of concentration (bottom). Samples were diluted to the indicated concentrations in 20 mM potassium phosphate buffer, pH 6.2, 10% D₂O.

FIG. 14 shows dynamic light scattering which indicates that BIF-44 does not aggregate in solution. While 4-ADPA (gray) and I4PTH (black) demonstrated dose-dependent increase in light scattering, the BIF-44 signal remains flat. Samples were diluted to the indicated concentrations in 20 mM potassium phosphate buffer, pH 6.2.

FIG. 15 shows ensemble docking to define the BIF-44 binding site on BAX. BIF-44 was docked to all 20 NMR solution structures (PDB:1F16) using the HSQC results to guide the docking. The pose with the best binding score for each model is shown. The BIF-44 binding pocket (bottom, black) is comprised of the following residues: Ile80, Ala81, Ala82, Va183, Asp84, Thr85, Asp86, Ser87, Pro88, Val91, Phe116, Lys119, Leu120, Val121, Lys123, Ala124, Thr127, Leu132, and Ile136.

### DETAILED DESCRIPTION

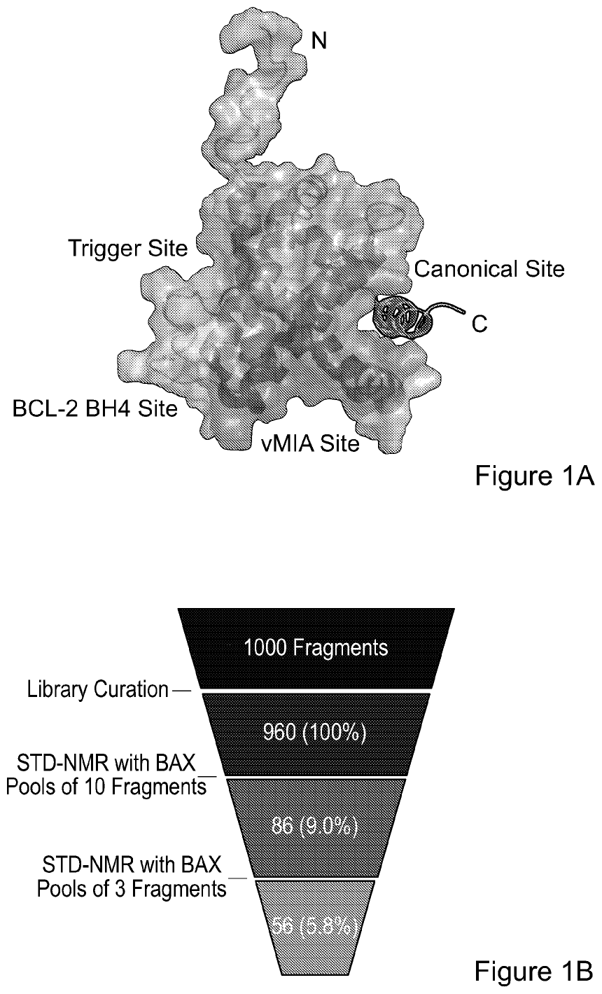

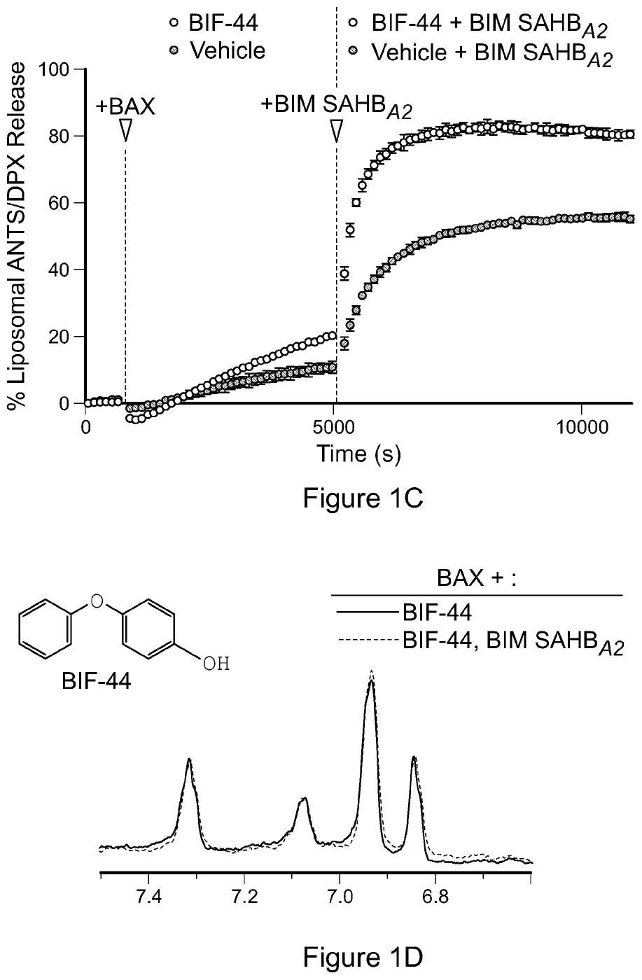

For such a small protein, a surprisingly large series of regulatory surfaces and complex conformational changes have been defined for BAX, as shown in FIG. 1a. In its conformationally inactive state, BAX is predominantly cytosolic and may also cycle to and from the mitochondrial outer membrane (MOM) region through a retrotranslocation process mediated by anti-apoptotic proteins, such as BCL-XL (see e.g, Edlich et al, Cell, 2011, 145:104-116). In response to stress, BH3-only direct activator proteins, such as BIM, BID, and PUMA, can directly and sequentially engage the α1/α6 trigger site and canonical hydrophobic groove to initiate and propagate BAX homo-oligomerization (see e.g., Czabotar et al, Cell, 2013, 152:519-531; Edwards et al, Chem. Biol. 2013, 20:888-902; Gavathiotis et al, Mol. Cell, 2010, 40:481-492; Gavathiotis et al, Nature, 2008, 455:1076-1081), whereas the BCL-2 canonical groove, the BCL-2 BH4 motif, and the cytomegalovirus vMIA protein can bind to and inhibit BAX (see e.g., Barclay et al, Mol. Cell, 2015, 57:873-886; Ma et al, Proc. Natl. Acad. Sci. U.S.A., 2012; 109:20901-20906; Petros et al, Proc. Natl. Acad. Sci. U.S.A., 2001, 98:3012-3017). BAX's central role in apoptosis induction derives from its capacity to undergo a major conformational change that results in irreversible mitochondrial translocation, intramembrane homo-oligomerization, and MOM poration (see e.g., Walensky et al, Trends Biochem. Sci. 2011, 36:642-652). The inherent risk to the cell of renegade BAX activation may underlie the mechanistic basis for its multifaceted regulation.

Given the central role of BCL-2 family proteins in apoptosis regulation during health and disease, efforts have been underway to disarm anti-apoptotic proteins in cancer, where sequestration and inactivation of pro-apoptotic members drives cellular immortality. Specifically, the mechanism by which anti-apoptotic proteins such as BCL-2 deploy a surface groove to trap the apoptosis-triggering BCL-2 homology 3 (BH3) helices of pro-apoptotic proteins, has now been drugged by venetoclax, a selective BCL-2 pocket inhibitor (see e.g., Souers et al, Nat. Med. 2013, 19:202-208; Sattler et al, Science, 1997, 275:983-986). This “inhibit the inhibitor” therapeutic strategy is being applied to develop drugs against the broad spectrum of anti-apoptotic targets implicated in cancer, including BCL-XL (see e.g., Lessene et al, Nat. Chem. Biol. 2013, 9:390-397; Tao et al, ACS Med. Chem. Lett. 2014, 5:1088-1093; Tse et al, Cancer Res. 2008, 68:3421-3428), MCL-1 (see e.g., Bruncko et al, J. Med. Chem. 2015, 58:2180-2194; Cohen et al, Chem. Biol. 2012, 19:1175-1186; Kotschy et al, Nature, 2016, 538:477-482; Leverson et al, Cell Death Dis. 2015, 6:e1590; Pelz et al, J. Med. Chem. 2016, 59:2054-2066; Stewart et al, Nat. Chem. Biol. 2010, 595-601), and BFL-1/A1 (see e.g., Huhn et al, Cell Chem. Biol. 2016, 23:1123-1134).

Having discovered an α1/α6 trigger site for direct BAX activation by pro-apoptotic BH3 domains, it was reasoned that an “activate the activators” strategy to drive cancer cell death also warranted therapeutic exploration (see e.g., Gavathiotis et al, Mol. Cell, 2010, 40:481-492; Gavathiotis et al, Nature, 2008, 455:1076-1081). Previous reports initiated this effort by in silico screening because, in contrast to the highly stable anti-apoptotic targets, the production of BAX for direct, experimental screening was hampered by the challenges in expressing sufficient quantities of recombinant BAX and the general instability of BAX in solution, especially when exposed to potential activators. The in silico and follow-up biochemical and cellular validation efforts yielded the first direct and selective BAX activator molecules (BAMs) (see e.g., Gavathiotis et al, Nat. Chem. Biol. 2012, 8:639-645). The present application provides BAX-activating compounds for potential clinical development by overcoming prior logistical challenges and directly executing a small molecule fragment screen by nuclear magnetic resonance (NMR) spectroscopy.

Accordingly, the present application provides compounds or molecular fragments that engage BAX at a deep hydrophobic pocket in a region that can otherwise be naturally occluded by the BAX-inhibitory BH4 domain of BCL-2 (see e.g., Barclay et al, Mol. Cell, 2015, 57:873-886) or cytomegalovirus vMIA peptide (see e.g., Ma et al, Proc. Natl. Acad. Sci. U.S.A. 2012, 109:20901-20906). In addition, the present application describes that molecular engagement sensitizes BAX by allosteric mobilization of the α1-α2 loop and the BAX BH3 helix, highlighting key mechanistic steps involved in BH3-mediated direct activation and homo-oligomerization of BAX (see e.g., Gavathiotis et al, Mol. Cell, 2010, 40:481-492; Wang et al, Mol. Cell. Biol. 1998, 18:6083-6089).

### Compounds and Compositions

The present application provides, inter alia, a composition, comprising a compound of Formula I:

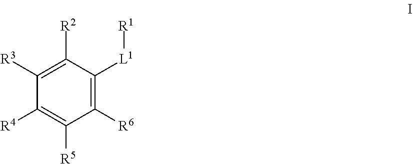

or a pharmaceutically acceptable salt thereof, and one or more pharmaceutically acceptable carriers, wherein L¹ is selected from the group consisting of a bond, C₁₋₃ alkylene, —O—, —O(C₁₋₃ alkylene)-, C₁₋₃ cyanoalkylene, —S—, —SO₂—, —S(C₁₋₃ alkylene)-, and —C(O)—; R¹ is selected from the group consisting of halo, OH, C₁₋₃ alkyl, C₁₋₃ haloalkyl, NH₂, CN, phenyl, 5-6 membered heteroaryl, and 5-6 membered heterocycloalkyl, wherein the phenyl, 5-6 membered heteroaryl, and 5-6 membered heterocycloalkyl are each optionally substituted by 1, 2, or 3 independently selected R^{A} groups; R² is selected from the group consisting of H, halo, OH, CN, C₁₋₃ alkyl, and C(O)OC₁₋₃ alkyl; R³ is selected from the group consisting of H, halo, OH, NH₂, C(O)C₁₋₃ alkyl, and C(S)C₁₋₃ alkyl; R⁴ is selected from the group consisting of H, halo, OH, NH₂, CN, C₁₋₃ alkyl, C₁₋₃ haloalkyl, and O(C₁₋₃ cyanoalkyl); R⁵ is selected from the group consisting of H, halo, OH, NH₂, and C(O)C₁₋₃ alkyl; R⁶ is selected from the group consisting of H, halo, OH, CN, C₁₋₃ alkyl, and C(O)OC₁₋₃ alkyl; and each R^{A} is independently selected from the group consisting of OH, NH₂, CN, C₁₋₃ alkyl, C₁₋₃ hydroxyalkyl, C(O)OH, C(O)C₁₋₃ alkyl, and C(O)N(C₁₋₃ alkyl)₂, wherein the C₁₋₃ alkyl group is optionally substituted by NH₂.

In some embodiments, L¹ of Formula I is selected from the group consisting of a bond, —CH₂—, —O—, —OCH₂—, —CH(CN)—, —S—, —SO₂—, —SCH₂—, and —C(O)—.

In some embodiments, L¹ of Formula I is —O—, —CH₂—, or —OCH₂—.

In some embodiments, R¹ of Formula I is selected from the group consisting of Cl, CH₃, CF₃, NH₂, CN, phenyl, 5-6 membered heteroaryl, and 5-6 membered heterocycloalkyl, wherein the phenyl, 5-6 membered heteroaryl, and 5-6 membered heterocycloalkyl are each optionally substituted by 1 or 2 independently selected R^{A} groups.

In some embodiments, R¹ of Formula I is selected from the group consisting of C₁, CH₃, CF₃, NH₂, CN, phenyl, pyridyl, furanyl, thienyl, pyrrolyl, thiazolyl, oxazolyl, pyrazolyl, 1,2,4-thiadiazolyl, piperidinyl, morpholinyl, and 4,5-dihydrothiazolyl wherein the phenyl, pyridyl, furanyl, thienyl, pyrrolyl, thiazolyl, oxazolyl, pyrazolyl, 1,2,4-thiadiazolyl, piperidinyl, morpholinyl, and 4,5-dihydrothiazolyl are each optionally substituted by 1 or 2 independently selected R^{A} groups.

In some embodiments, each R^{A} of Formula I is independently selected from the group consisting of OH, NH₂, CN, CH₃, CH₂OH, CH₂CH₂NH₂, C(O)OH, C(O)CH₃, and C(O)N(CH₃)₂.

In some embodiments, R¹ of Formula I is phenyl which is optionally substituted by 1 or 2 independently selected R^{A} groups.

In some embodiments, R¹ of Formula I is phenyl which is optionally substituted by 1 or 2 independently selected R^{A} groups, wherein each R^{A} is independently selected from the group consisting of OH, NH₂, CH₂OH, and C(O)OH.

In some embodiments, R¹ of Formula I is phenyl, 4-hydroxyphenyl, 3-hydroxyphenyl, 4-aminophenyl, 4-carboxylphenyl, or 4-hydroxymethylphenyl.

In some embodiments, R² of Formula I is selected from the group consisting of H, Cl, CN, CH₃, and C(O)OCH₃.

In some embodiments, R² of Formula I is H or CH₃.

In some embodiments, R³ of Formula I is selected from the group consisting of H, F, Cl, NH₂, C(O)CH₃, and C(S)CH₃.

In some embodiments, R³ of Formula I is H.

In some embodiments, R⁴ of Formula I is selected from the group consisting of H, Cl, NH₂, CN, CH₃, CF₃, and OCH₃CN.

In some embodiments, R⁴ of Formula I is H or OH.

In some embodiments, R⁵ of Formula I is selected from the group consisting of H, F, Cl, NH₂, and C(O)CH₃.

In some embodiments, R⁵ of Formula I is H or NH₂.

In some embodiments, R⁶ of Formula I is selected from the group consisting of H, Cl, CN, CH₃, and C(O)OCH₃.

In some embodiments, R⁶ of Formula I is H.

In some embodiments, L¹ of Formula I is selected from the group consisting of a bond, —CH₂—, —O—, —OCH₂—, —CH(CN)—, —S—, —SO₂—, —SCH₂—, and —C(O)—; R¹ of Formula I is selected from the group consisting of Cl, CH₃, CF₃, NH₂, CN, phenyl, 5-6 membered heteroaryl, and 5-6 membered heterocycloalkyl, wherein the phenyl, 5-6 membered heteroaryl, and 5-6 membered heterocycloalkyl are each optionally substituted by 1 or 2 independently selected R^{A} groups; each R^{A} of Formula I is independently selected from the group consisting of OH, NH₂, CN, CH₃, CH₂OH, CH₂CH₂NH₂, C(O)OH, C(O)CH₃, and C(O)N(CH₃)₂; R² of Formula I is selected from the group consisting of H, Cl, CN, CH₃, and C(O)OCH₃; R³ of Formula I is selected from the group consisting of H, F, Cl, NH₂, C(O)CH₃, and C(S)CH₃; R⁴ of Formula I is selected from the group consisting of H, Cl, NH₂, CN, CH₃, CF₃, and OCH₃CN; R⁵ of Formula I is selected from the group consisting of H, F, Cl, NH₂, and C(O)CH₃; and R⁶ of Formula I is selected from the group consisting of H, Cl, CN, CH₃, and C(O)OCH₃.

In some embodiments, L¹ of Formula I is selected from the group consisting of a bond, —CH₂—, —O—, —OCH₂—, —CH(CN)—, —S—, —SO₂—, —SCH₂—, and —C(O)—; R¹ of Formula I is phenyl which is optionally substituted by 1 or 2 independently selected R^{A} groups; each R^{A} of Formula I is independently selected from the group consisting of OH, NH₂, CN, CH₃, CH₂OH, CH₂CH₂NH₂, C(O)OH, C(O)CH₃, and C(O)N(CH₃)₂; R² of Formula I is selected from the group consisting of H, Cl, CN, CH₃, and C(O)OCH₃; R³ of Formula I is selected from the group consisting of H, F, Cl, NH₂, C(O)CH₃, and C(S)CH₃; R⁴ of Formula I is selected from the group consisting of H, Cl, NH₂, CN, CH₃, CF₃, and OCH₃CN; R⁵ of Formula I is selected from the group consisting of H, F, Cl, NH₂, and C(O)CH₃; and R⁶ of Formula I is selected from the group consisting of H, Cl, CN, CH₃, and C(O)OCH₃.

In some embodiments, the compound of Formula I is selected from the group consisting of:

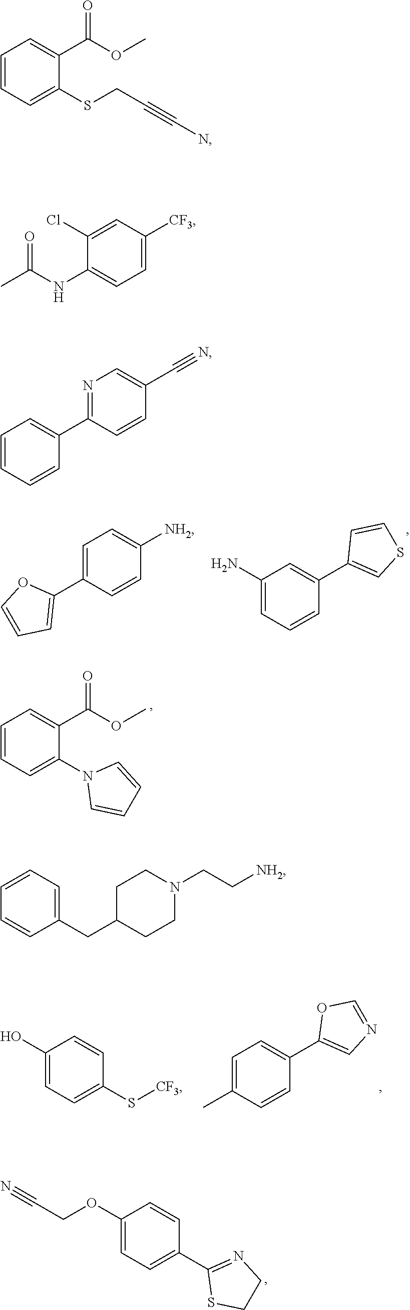

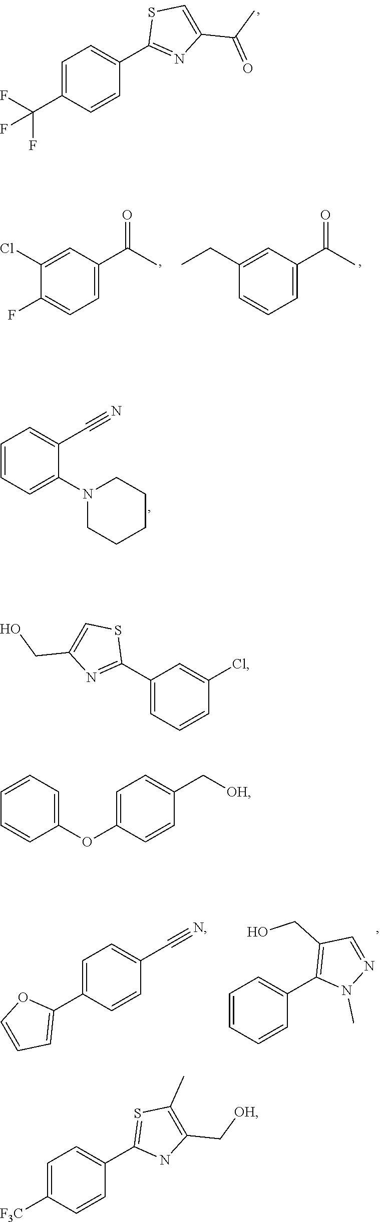

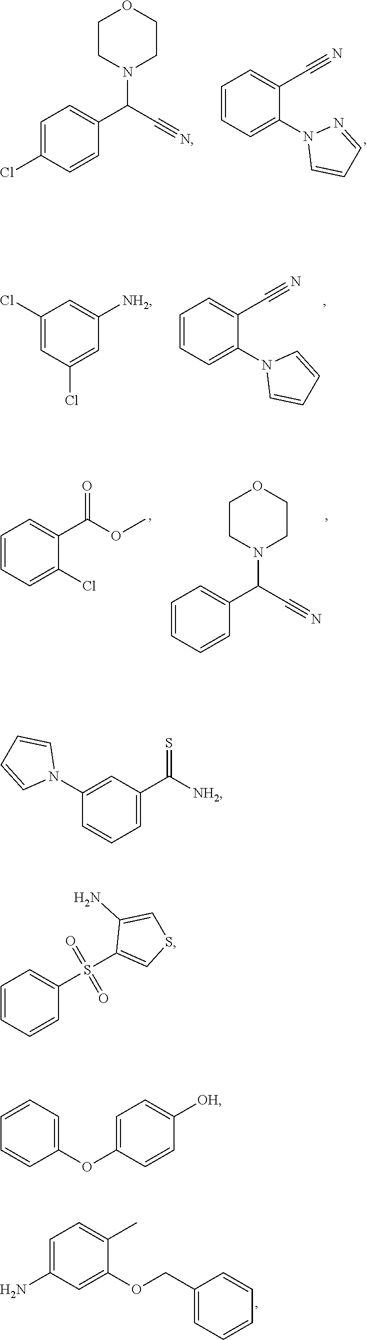

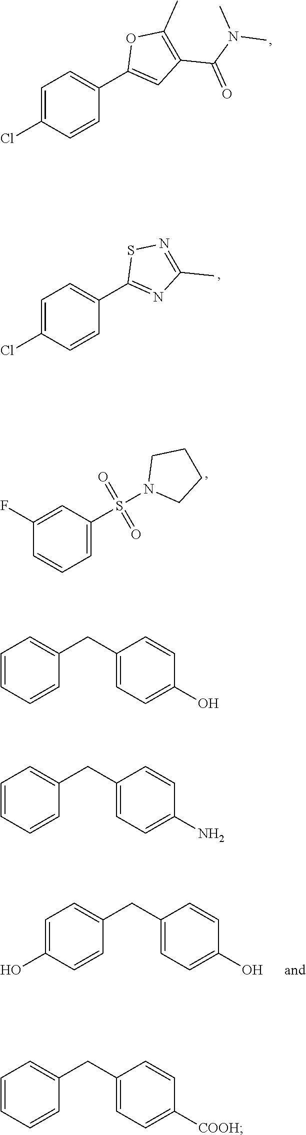

or a pharmaceutically acceptable salt thereof.

In some embodiments, the compound of Formula I is:

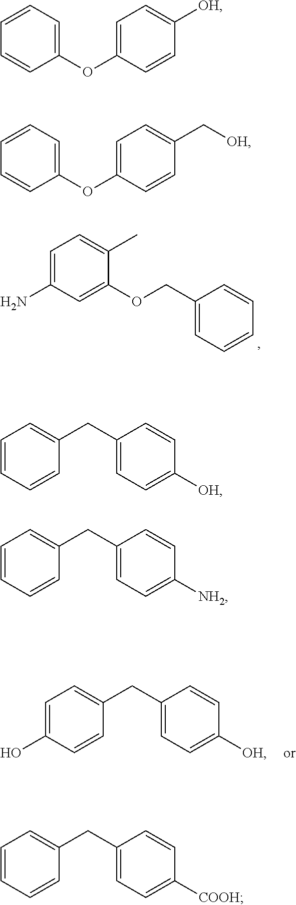

or a pharmaceutically acceptable salt thereof.

In some embodiments, the compound of Formula I is:

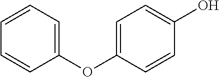 [SMILES: Oc1ccc(Oc2ccccc2)cc1]

or a pharmaceutically acceptable salt thereof.

The present application further provides a composition, comprising a compound of Formula II:

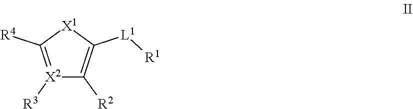

or a pharmaceutically acceptable salt thereof, and one or more pharmaceutically acceptable carriers, wherein X¹ is NH or S; X² is C or N; L¹ is selected from the group consisting of a bond, —C(O)—, —C(O)O—, and —SO₂—; R¹ is selected from the group consisting of C₁₋₃ alkyl, NH₂, di(C₁₋₃ alkyl)amino, and a 5-6 membered heterocycloalkyl; R² is selected from the group consisting of H, halo, C₁₋₃ alkyl, and C(O)OC₁₋₃ alkyl; R³ is selected from the group consisting of H, C₁₋₃ alkyl, and 5-6 membered heteroaryl; or R³ is absent when X² is N; and R⁴ is selected from the group consisting of H and C₁₋₃ alkyl.

In some embodiments, X¹ of Formula II is NH.

In some embodiments, X¹ of Formula II is S.

In some embodiments, X² of Formula II is C.

In some embodiments, X² of Formula II is N.

In some embodiments, X¹ of Formula II is NH and X² of Formula II is C.

In some embodiments, X¹ of Formula II is NH and X² of Formula II is N.

In some embodiments, X¹ of Formula II is S and X² of Formula II is C.

In some embodiments, X¹ of Formula II is S and X² of Formula II is N.

In some embodiments, R¹ of Formula II is selected from the group consisting of CH₃, CH₂CH₃, NH₂, N(CH₂CH₃)₂, piperidinyl, and dihydrothiophen-3(2H)-onyl.

In some embodiments, -L¹-R¹ of Formula II forms a group selected from the group consisting of NH₂, C(O)OCH₃, C(O)OCH₂CH₃, C(O)N(CH₂CH₃)₂, SO₀₂-piperidinyl, and dihydrothiophen-3(2H)-onyl.

In some embodiments, R² of Formula II is selected from the group consisting of H, Cl, CH₃, and C(O)OCH₂CH₃.

In some embodiments, R³ of Formula II is selected from the group consisting of H, CH₃, CH₂CH₃, and thienyl.

In some embodiments, R⁴ of Formula II is selected from the group consisting of H and C₁₋₃ alkyl.

In some embodiments, X¹ of Formula II is NH or S; X² of Formula II is C or N; L¹ of Formula II is selected from the group consisting of a bond, —C(O)—, —C(O)O—, and —SO₂—; R¹ of Formula II is selected from the group consisting of CH₃, CH₂CH₃, NH₂, N(CH₂CH₃)₂, piperidinyl, and dihydrothiophen-3(2H)-onyl; R² of Formula II is selected from the group consisting of H, Cl, CH₃, and C(O)OCH₂CH₃; R³ of Formula II is selected from the group consisting of H, CH₃, CH₂CH₃, and thienyl; and R⁴ of Formula II is selected from the group consisting of H and C₁₋₃ alkyl.

In some embodiments, X¹ of Formula II is NH or S; X² of Formula II is C or N; -L¹-R¹ of Formula II forms a group selected from the group consisting of NH₂, C(O)OCH₃, C(O)OCH₂CH₃, C(O)N(CH₂CH₃)₂, SO₂-piperidinyl, and dihydrothiophen-3(2H)-onyl; R² of Formula II is selected from the group consisting of H, Cl, CH₃, and C(O)OCH₂CH₃; R³ of Formula II is selected from the group consisting of H, CH₃, CH₂CH₃, and thienyl; and R⁴ of Formula II is selected from the group consisting of H and C₁₋₃ alkyl.

In some embodiments, the compound of Formula II is selected from the group consisting of:

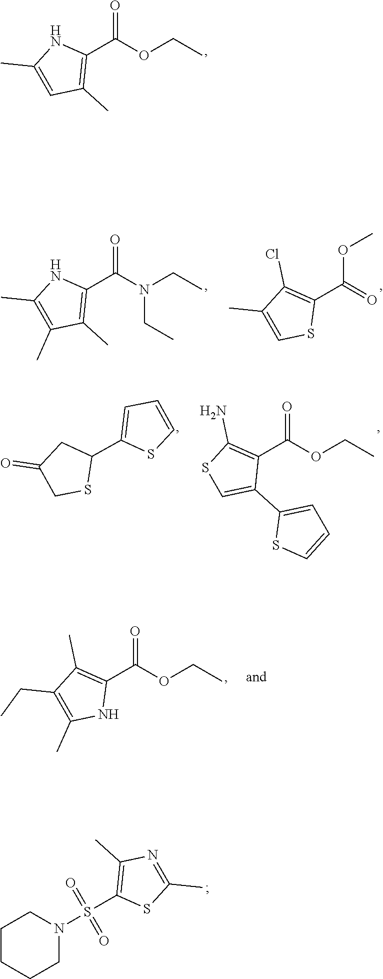

or a pharmaceutically acceptable salt thereof.

The present application further provides a composition, comprising a compound of Formula III:

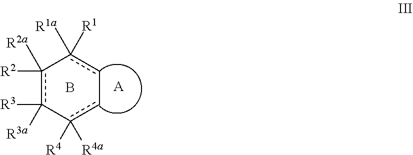

or a pharmaceutically acceptable salt thereof, and one or more pharmaceutically acceptable carriers, wherein, [symbol] refers to a single bond or a double bond; Ring A forms a fused ring with Ring B and Ring A is selected from the group consisting of a 5-6 membered cycloalkyl, a 5-6 membered heteroaryl, and a 5-6 membered heterocycloalkyl, wherein Ring A is optionally substituted by 1, 2, or 3 independently selected R^{A} groups; R¹ is selected from the group consisting of H, C(O)OC₁₋₃ alkyl, OC(O)C₁₋₃ alkyl, C(S)NH₂, and ═N—OH; R^{1a} is H; or R^{1a} is absent when the carbon atom to which R^{1a} is attached forms a double bond; R² is selected from the group consisting of H and halo; R^{2a} is H; or R^{2a} is absent when the carbon atom to which R^{2a} is attached forms a double bond; R³ is selected from the group consisting of H, halo, C₁₋₃ alkyl, C₁₋₃ hydroxyalkyl, NHC(O)C₁₋₃ alkyl, and (C₁₋₃ alkylene)NHC₁₋₃ alkyl; R^{3a} is C₁₋₃ alkyl; or R^{3a} is absent when the carbon atom to which R^{3a} is attached forms a double bond; R⁴ is selected from the group consisting of H and C₁₋₃ alkyl; R^{4a} is H; or R^{4a} is absent when the carbon atom to which R^{4a} is attached forms a double bond; and each R^{A} is independently selected from the group consisting of ═O, ═S, CN, C₁₋₃ alkyl, C₁₋₃ hydroxyalkyl, S(C₁₋₃ alkyl), and C(O)OH.

In some embodiments, Ring A is a 5-6 membered heteroaryl which is optionally substituted by 1, 2, or 3 independently selected R^{A} groups.

In some embodiments, Ring A is a 5-6 membered heterocycloalkyl which is optionally substituted by 1, 2, or 3 independently selected R^{A} groups.

In some embodiments, each R^{A} of Formula III is independently selected from the group consisting of ═O, ═S, CN, CH₃, CH₂OH, SCH₃, and C(O)OH.

In some embodiments, Ring A is an unsubstituted 5-6 membered cycloalkyl.

In some embodiments, Ring A is selected from the group consisting of:

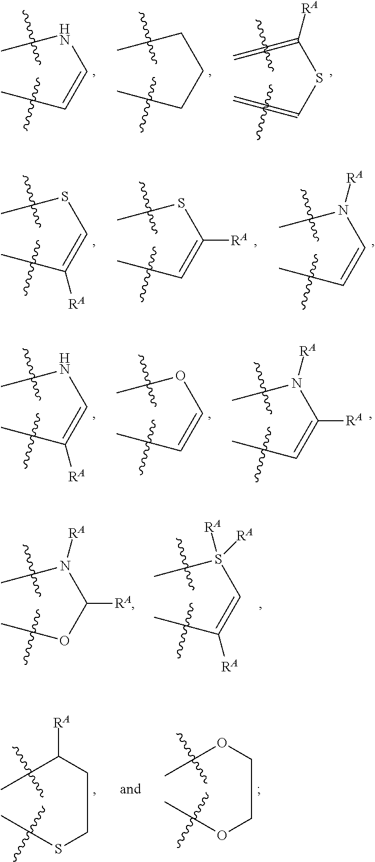

wherein each [symbol] indicates the bonds connecting the fused Ring A and Ring B.

In some embodiments, R¹ of Formula III is selected from the group consisting of H, C(O)OCH₃, OC(O)CH₃, C(S)NH₂, and ═N—OH.

In some embodiments, R² of Formula III is selected from the group consisting of H and Cl.

In some embodiments, R^{2a} of Formula III is H.

In some embodiments, R^{2a} of Formula III is absent.

In some embodiments, R³ of Formula III is selected from the group consisting of H, Cl, CH₃, CH₂OH, NHC(O)CH₃, and CH₂NHCH₃.

In some embodiments, R^{3a} of Formula III is CH₃.

In some embodiments, R^{3a} of Formula III is absent.

In some embodiments, R⁴ of Formula III is selected from the group consisting of H and CH₃.

In some embodiments, Ring A is selected from the group consisting of a 5-6 membered heteroaryl, a 5-6 membered heterocycloalkyl, and an unsubstituted 5-6 membered cycloalkyl, wherein the 5-6 membered heteroaryl and 5-6 membered heterocycloalkyl group are each optionally substituted by 1, 2, or 3 independently selected R^{A} groups; R¹ of Formula III is selected from the group consisting of H, C(O)OCH₃, OC(O)CH₃, C(S)NH₂, and ═N—OH; R² of Formula III is selected from the group consisting of H and Cl; R^{2a} of Formula III is H; or R^{2a} is absent when the carbon atom to which R^{2a} is attached forms a double bond; R³ of Formula III is selected from the group consisting of H, Cl, CH₃, CH₂OH, NHC(O)CH₃, and CH₂NHCH₃; R^{3a} of Formula III is C₁₋₃ alkyl; or R^{3a} is absent when the carbon atom to which R^{3a} is attached forms a double bond; R⁴ of Formula III is selected from the group consisting of H and CH₃; R^{4a} of Formula III is H; or R^{4a} is absent when the carbon atom to which R^{4a} is attached forms a double bond; and each R^{A} of Formula III is independently selected from the group consisting of ═O, ═S, CN, CH₃, CH₂OH, SCH₃, and C(O)OH.

In some embodiments, the compound of Formula III is selected from the group consisting of:

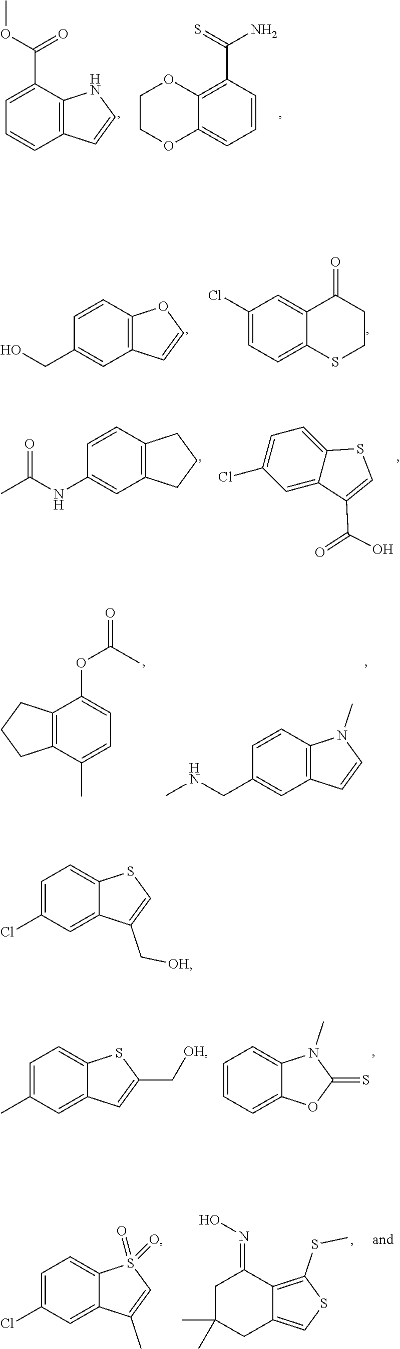

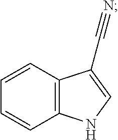 [SMILES: N#Cc1c[nH]c2ccccc12]

or a pharmaceutically acceptable salt thereof.

The present application further provides a composition, comprising a compound comprising a moiety of Formula IV:

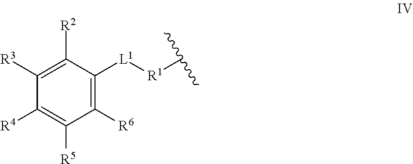

or a pharmaceutically acceptable salt thereof, and one or more pharmaceutically acceptable carriers, wherein L¹ is selected from the group consisting of a bond, C₁₋₃ alkylene, —O—, —O(C₁₋₃ alkylene)-, C₁₋₃ cyanoalkylene, —S—, —SO₂—, —S(C₁₋₃ alkylene)-, and —C(O)—; R¹ is selected from the group consisting of phenylene, 5-6 membered heteroarylene, and 5-6 membered heterocycloalkylene, each of which is optionally substituted by 1, 2, or 3 independently selected R^{A} groups; R² is selected from the group consisting of H, halo, OH, CN, C₁₋₃ alkyl, and C(O)OC₁₋₃ alkyl; R³ is selected from the group consisting of H, halo, OH, NH₂, C(O)C₁₋₃ alkyl, and C(S)C₁₋₃ alkyl; R⁴ is selected from the group consisting of H, halo, OH, NH₂, CN, C₁₋₃ alkyl, C₁₋₃ haloalkyl, and O(C₁₋₃ cyanoalkyl); R⁵ is selected from the group consisting of H, halo, OH, NH₂, and C(O)C₁₋₃ alkyl; R⁶ is selected from the group consisting of H, halo, OH, CN, C₁₋₃ alkyl, and C(O)OC₁₋₃ alkyl; and each R^{A} is independently selected from the group consisting of OH, NH₂, CN, C₁₋₃ alkyl, C₁₋₃ hydroxyalkyl, C(O)OH, C(O)C₁₋₃ alkyl, and C(O)N(C₁₋₃ alkyl)₂, wherein the C₁₋₃ alkyl group is optionally substituted by NH₂.

In some embodiments, L¹ of Formula IV is selected from the group consisting of a bond, —CH₂—, —O—, —OCH₂—, —CH(CN)—, —S—, —SO₂—, —SCH₂—, and —C(O)—.

In some embodiments, R¹ of Formula IV is phenylene optionally substituted by 1 or 2 independently selected R^{A} groups.

In some embodiments, each R^{A} of Formula IV is independently selected from the group consisting of OH, NH₂, CN, CH₃, CH₂OH, CH₂CH₂NH₂, C(O)OH, C(O)CH₃, and C(O)N(CH₃)₂.

In some embodiments, R² of Formula IV is selected from the group consisting of H, Cl, CN, CH₃, and C(O)OCH₃.

In some embodiments, R³ of Formula IV is selected from the group consisting of H, F, Cl, NH₂, C(O)CH₃, and C(S)CH₃.

In some embodiments, R⁴ of Formula IV is selected from the group consisting of H, Cl, NH₂, CN, CH₃, CF₃, and OCH₃CN.

In some embodiments, R⁵ of Formula IV is selected from the group consisting of H, F, Cl, NH₂, and C(O)CH₃.

In some embodiments, R⁶ of Formula IV is selected from the group consisting of H, Cl, CN, CH₃, and C(O)OCH₃.

In some embodiments, L¹ of Formula IV is selected from the group consisting of a bond, —CH₂—, —O—, —OCH₂—, —CH(CN)—, —S—, —SO₂—, —SCH₂—, and —C(O)—; R¹ of Formula IV is phenylene optionally substituted by 1 or 2 independently selected R^{A} groups; each R^{A} of Formula IV is independently selected from the group consisting of OH, NH₂, CN, CH₃, CH₂OH, CH₂CH₂NH₂, C(O)OH, C(O)CH₃, and C(O)N(CH₃)₂; R² of Formula IV is selected from the group consisting of H, Cl, CN, CH₃, and C(O)OCH₃; R³ of Formula IV is selected from the group consisting of H, F, Cl, NH₂, C(O)CH₃, and C(S)CH₃; R⁴ of Formula IV is selected from the group consisting of H, Cl, NH₂, CN, CH₃, CF₃, and OCH₃CN; R⁵ of Formula IV is selected from the group consisting of H, F, Cl, NH₂, and C(O)CH₃; and R⁶ of Formula IV is selected from the group consisting of H, Cl, CN, CH₃, and C(O)OCH₃.

When employed as pharmaceuticals, the compositions provided herein can be administered in the form of pharmaceutical compositions. These compositions can be prepared as described herein or elsewhere, and can be administered by a variety of routes, depending upon whether local or systemic treatment is desired and upon the area to be treated. Administration may be topical (including transdermal, epidermal, ophthalmic and to mucous membranes including intranasal, vaginal, and rectal delivery), pulmonary (e.g., by inhalation or insufflation of powders or aerosols, including by nebulizer; intratracheal or intranasal), oral, or parenteral. Parenteral administration includes intravenous, intraarterial, subcutaneous, intraperitoneal intramuscular injection, or intraperitoneal intramuscular infusion; or intracranial, (e.g., intrathecal or intraventricular, administration). Parenteral administration can be in the form of a single bolus dose, or may be, for example, by a continuous perfusion pump.

Pharmaceutical compositions for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Also provided are compositions which contain, as the active ingredient, a compound provided herein (e.g., a compound of Formulas I-III or a compound comprising a moiety of Formula IV), or a pharmaceutically acceptable salt thereof, in combination with one or more pharmaceutically acceptable carriers (e.g., excipients). In preparing the compositions provided herein, the active ingredient is typically mixed with an excipient, diluted by an excipient, or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments, soft and hard gelatin capsules, suppositories, sterile injectable solutions, and sterile packaged powders.

Examples of suitable excipients include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The compositions can additionally include, without limitation, lubricating agents such as talc, magnesium stearate, and mineral oil; wetting agents; emulsifying and suspending agents; preserving agents such as methyl- and propylhydroxy-benzoates; sweetening agents; flavoring agents, or combinations thereof.

The active ingredient can be effective over a wide dosage range and is generally administered in a therapeutically effective amount. It will be understood, however, that the amount of the compound and/or composition actually administered will usually be determined by a physician, according to the relevant circumstances, including the condition to be treated, the chosen route of administration, the actual compound or composition administered, the age, weight, and response of the individual subject, the severity of the subject's symptoms, and the like.

At various places in the present specification, divalent linking substituents are described. It is specifically intended that each divalent linking substituent include both the forward and backward forms of the linking substituent. For example, —NR(CR′R″)ₙ— includes both —NR(CR′R″)ₙ— and —(CR′R″)ₙNR—. Where the structure clearly requires a linking group, the Markush variables listed for that group are understood to be linking groups.

As used herein, the phrase “optionally substituted” means unsubstituted or substituted. As used herein, the term “substituted” means that a hydrogen atom is removed and replaced by a substituent. It is to be understood that substitution at a given atom is limited by valency.

Throughout the definitions, the term “Cₙ₋ₘ” indicates a range which includes the endpoints, wherein n and m are integers and indicate the number of carbons. Examples include C₁₋₄, C₁₋₆, and the like.

As used herein, the term “Cₙ₋ₘ alkylene”, employed alone or in combination with other terms (e.g., cyanoalkylene), refers to a divalent alkyl linking group having n to m carbons. Examples of alkylene groups include, but are not limited to, methylene, ethan-1,2-diyl, propan-1,3-diyl, propan-1,2-diyl, and the like. In some embodiments, the alkylene moiety contains 1 to 3 carbon atoms or 1 to 2 carbon atoms.

As used herein, the term “Cₙ₋ₘ cyanoalkylene” refers to a divalent alkyl linking group having n to m carbons, wherein the alkyl linking group is substituted by one or more cyano (i.e., —CN) groups. In some embodiments, the cyanoalkylene group contains 1 cyano group.

As used herein, the term “Cₙ₋ₘ alkyl”, employed alone or in combination with other terms, refers to a saturated hydrocarbon group that may be straight-chain or branched, having n to m carbons. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains 1 to 3 carbon atoms or 1 to 2 carbon atoms.

As used herein, “halo” refers to F, Cl, Br, or I. In some embodiments, the halo is F, Cl, or Br. In some embodiments, the halo is F or Cl.

As used herein, the term “Cₙ₋ₘhaloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group contains 1 to 3 carbon atoms or 1 to 2 carbon atoms. In some embodiments, the haloalkyl group contains 1 halo group.

As used herein, the term “Cₙ₋ₘhydroxyalkyl”, employed alone or in combination with other terms, refers to an alkyl group having from one hydroxy group (i.e., —OH) to 2s+1 hydroxy groups, where “s” is the number of carbon atoms in the alkyl group, wherein the alkyl group has n to m carbon atoms. In some embodiments, the hydroxyalkyl group contains 1 to 3 carbon atoms or 1 to 2 carbon atoms. In some embodiments, the hydroxyalkyl group contains 1 hydroxy group.

As used herein, the term “Cₙ₋ₘ cyanoalkyl”, employed alone or in combination with other terms, refers to an alkyl group having from one cyano group (i.e., —CN) to 2s+1 cyano groups, where “s” is the number of carbon atoms in the alkyl group, wherein the alkyl group has n to m carbon atoms. In some embodiments, the cyanoalkyl group contains 1 to 3 carbon atoms or 1 to 2 carbon atoms. In some embodiments, the cyanoalkyl group contains 1 cyano group.

As used herein, the term “di(Cₙ₋ₘ-alkyl)amino” refers to a group of formula —N(alkyl)₂, wherein the two alkyl groups each have, independently, n to m carbon atoms. In some embodiments, each alkyl group independently has 1 to 3 carbon atoms or 1 to 2 carbon atoms.

As used herein, “cycloalkyl” refers to non-aromatic cyclic hydrocarbons including cyclized alkyl and/or alkenyl groups. Cycloalkyl groups can include mono- or polycyclic (e.g., having 2, 3 or 4 fused rings) groups and spirocycles. Cycloalkyl groups can have 3, 4, 5, or 6 ring-forming carbons (i.e., a C₃₋₆ cycloalkyl group). Ring-forming carbon atoms of a cycloalkyl group can be optionally substituted by oxo or sulfido (e.g., ═O or ═S). Example cycloalkyl groups include cyclopropyl, cyclobutyl, cyclopentyl, cyclohexyl, and the like. In some embodiments, the cycloalkyl has 3-6 ring-forming carbon atoms (i.e., a C₃₋₆ cycloalkyl group).

As used herein, the term “heteroaryl” refers to an aromatic mono- or polycyclic heterocycle having at least one heteroatom ring member selected from sulfur, oxygen, and nitrogen. In some embodiments, the heteroaryl ring has 1, 2, 3, or 4 heteroatom ring members independently selected from nitrogen, sulfur, and oxygen. In some embodiments, the heteroaryl has 5-6 ring atoms and 1, 2, 3, or 4 heteroatom ring members independently selected from nitrogen, sulfur and oxygen. Exemplary five-membered ring heteroaryls include, but are not limited to, thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl. Exemplary six-membered ring heteroaryls include, but are not limited to, pyridyl, pyrazinyl, pyrimidinyl, triazinyl and pyridazinyl.

As used herein, the term “heterocycloalkyl” refers to non-aromatic monocyclic or polycyclic heterocycles having 1, 2, 3, or 4 ring-forming heteroatoms selected from O, N, or S. Included in heterocycloalkyl are monocyclic 4-, 5-, and 6-membered heterocycloalkyl groups. Exemplary heterocycloalkyl groups include, pyranyl, oxetanyl, azetidinyl, morpholinyl, thiomorpholinyl, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, and the like. Ring-forming carbon atoms and heteroatoms of a heterocycloalkyl group can be optionally substituted by oxo or sulfido (e.g., ═O, ═S). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the cycloalkyl ring, for example, benzo or thienyl derivatives of piperidine, morpholine, azepine, and the like. A heterocycloalkyl group containing a fused aromatic ring can be attached through any ring-forming atom including a ring-forming atom of the fused aromatic ring. In some embodiments, the heterocycloalkyl has 5-6 ring atoms with 1 or 2 heteroatoms independently selected from nitrogen, oxygen, or sulfur.

As used herein, the term “phenylene” refers to a divalent phenyl linking group.

As used herein, the term “heteroarylene” refers to a divalent heteroaryl linking group. In some embodiments, the heteroarylene has 5-6 ring atoms.

As used herein, the term “heterocycloalkylene” refers to a divalent heterocycloalkyl linking group. In some embodiments, the heterocycloalkylene has 5-6 ring atoms.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas a pyridin-3-yl ring is attached at the 3-position.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Exemplary prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

The phrase “pharmaceutically acceptable” is employed herein to refer to those compounds, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present application also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present application include the conventional non-toxic salts of the parent compound formed, for example, from non-toxic inorganic or organic acids. The pharmaceutically acceptable salts of the present application can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (MeCN) are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977). Conventional methods for preparing salt forms are described, for example, in Handbook of Pharmaceutical Salts: Properties, Selection, and Use, Wiley-VCH, 2002.

### Methods of Use

The present application further provides a method of sensitizing and/or activating pro-apoptotic activity of BAX. In some embodiments, the method comprises contacting a cell sample or tissue sample comprising BAX with a composition provided herein (e.g., a composition comprising a compound of Formulas I-III or a compound comprising a moiety of Formula IV, or a pharmaceutically acceptable salt thereof). As used herein, the term “contacting” refers to the bringing together of indicated components in an in vitro system. For example, “contacting” a BAX polypeptide with a composition provided herein includes introducing a compound of the invention into a sample (e.g., a cell sample or tissue sample) containing a cellular or purified preparation containing the BAX polypeptide. In some embodiments, the composition comprising a compound of Formulas I-III or the compound comprising a moiety of Formula IV sensitizes activation of the pro-apoptotic activity of the BAX polypeptide by another pro-apoptotic agent (i.e., enhancing the pro-apoptotic activity of the BAX polypeptide induced by the pro-apoptotic agent) in the cell sample or tissue sample. In such embodiments, the composition described herein may or may not itself activate the pro-apoptotic activity of the BAX polypeptide. In some embodiments, the composition comprising a compound of Formulas I-III or the compound comprising a moiety of Formula IV activates the pro-apoptotic activity of the BAX polypeptide in the cell sample or tissue sample. In such embodiments, the composition can be administered either in the presence or in the absence of another pro-apoptotic agent.

In some embodiments, the present application provides a method of sensitizing and/or activating pro-apoptotic activity of BAX in a subject. In some embodiments, the method comprises administering to the subject a compound or composition provided herein. In some embodiments, the compound or composition provided herein sensitizes activation of the pro-apoptotic activity of BAX in the subject (e.g., when the composition is administered in combination with another pro-apoptotic agent). In some embodiments, the compound or composition provided herein activates the pro-apoptotic activity of BAX in the subject (e.g., when the compositions is administered in the presence or absence of another pro-apoptotic agent). As used herein, the term “subject,” refers to any animal, including mammals. Examples of subjects include, but are not limited to, mice, rats, rabbits, dogs, cats, swine, cattle, sheep, horses, primates, and humans. In some embodiments, the subject is a human. In some embodiments, the method comprises administering to the subject a therapeutically effective amount of a composition provided herein. As used herein, the phrase “therapeutically effective amount” refers to the amount of active compound or pharmaceutical agent that elicits the biological or medicinal response that is being sought in a tissue, system, animal, individual or human by a researcher, veterinarian, medical doctor, or other clinician.

The present application further provides a method of treating cancer in a subject. In some embodiments, the method comprises administering to a subject in need of such treatment a therapeutically effective amount of a composition provided herein.

Exemplary cancers include, but are not limited to, breast cancer, prostate cancer, lymphoma, skin cancer, pancreatic cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma.

Exemplary leukemias and lymphomas include, but are not limited to, erythroblastic leukemia, acute megakaryoblastic leukemia, acute lymphocytic leukemia, acute promyeloid leukemia (APML), acute granulocytic leukemia, acute myelogenous leukemia (AML), chronic myelogenous leukemia (CML), acute lymphoblastic leukemia (ALL) (e.g., B-lineage ALL and T-lineage ALL), chronic lymphocytic leukemia (CLL), chronic granulocytic leukemia, prolymphocytic leukemia (PLL), hairy cell leukemia (HLL), Waldenstrom's macroglobulinemia (WM), non-Hodgkin lymphoma, peripheral T cell lymphomas, adult T cell leukemia/lymphoma (ATL), cutaneous T-cell lymphoma (CTCL), large granular lymphocytic leukemia (LGF), Hodgkin's disease, and Reed-Stemberg disease.

In some embodiments, the leukemia is selected from the group consisting of acute lymphocytic leukemia, chronic lymphocytic leukemia, chronic granulocytic leukemia, acute granulocytic leukemia, acute myelogenous leukemia, chronic myelogenous leukemia, and hairy cell leukemia.

In some embodiments, the leukemia is selected from the group consisting of acute lymphoblastic leukemia, acute myelogenous leukemia, chronic lymphoblastic leukemia, and chronic myelogenous leukemia.

The present application further provides a method for identifying a compound which activates the pro-apoptotic activity of a BAX polypeptide. In some embodiments, the method comprises:

a) contacting a binding site of said BAX polypeptide comprising an amino acid sequence of SEQ ID NO:1 with a compound in vitro under conditions suitable for activating the pro-apoptotic activity of the BAX polypeptide; and

b) determining whether the compound binds to one or more amino acid residues selected from the group consisting of Ile80, Ala81, Ala82, Va183, Asp84, Thr85, Asp86, Ser87, Pro88, Val91, Phe116, Lys119, Leu120, Val121, Lys123, Ala124, Thr127, Leu132, Ile136;

wherein the binding site of the BAX polypeptide comprises the junction of the α3-α4 and α5-α6 hairpins of the BAX polypeptide.

In some embodiments, the determining step is performed by saturation transfer difference NMR, HSQC NMR, surface plasmon resonance, biolayer interferometry, or competitive fluorescence polarization assay.

In some embodiments, binding of the compound to the BAX polypeptide causes a change in the signal of the NMR spectrum of the compound.

In some embodiments, the method further comprises detecting activation of the BAX polypeptide by the compound.

In some embodiments, the detecting step comprises performing an assay selected from the group consisting of detecting BAX oligomerization, antibody-based detection of BAX conformers, a mitochondrial cytochrome c release assay, a liposomal release assay, a cell death assay, a mitochondrial or cellular morphology assay, a mitochondrial calcium flux assay, a mitochondrial transmembrane quantitation assay, and quantitation of caspase 3 activity or annexin V binding.

In some embodiments, said compound binds to said binding site with an affinity of <1 mM, for example, <750 nM, <500 nM, <250 nM, <100 nM, <50 nM, <25 nM, <10 nM, and the like.

In some embodiments, the methods provided herein further comprise administering one or more additional therapeutic agents (e.g., chemotherapeutic agents) and/or performing one or more additional medical techniques (e.g., radiation therapies, surgical interventions, and the like) to a subject, in vitro cell samples, tissue samples, and/or organ samples.

In some embodiments, the methods further comprise administering one or more additional therapeutic agents selected from the group consisting of: agents that induce apoptosis; polynucleotides (e.g., anti-sense, ribozymes, siRNA); polypeptides (e.g., enzymes and antibodies); biological mimetics (e.g., BH3 mimetics); agents that bind to and inhibit anti-apoptotic proteins (e.g., agents that inhibit anti-apoptotic BCL-2 proteins); alkaloids; alkylating agents; antitumor antibiotics; antimetabolites; hormones; platinum compounds; monoclonal or polyclonal antibodies (e.g., antibodies conjugated with anticancer drugs, toxins, defensins, and the like), toxins, radionuclides; biological response modifiers (e.g., interferons such as IFN-α and the like) and interleukins (e.g., IL-2 and the like); adoptive immunotherapy agents; hematopoietic growth factors; agents that induce tumor cell differentiation (e.g., all-trans-retinoic acid and the like); gene therapy reagents (e.g., antisense therapy reagents and nucleotides); tumor vaccines; angiogenesis inhibitors; proteosome inhibitors: NF kappa.beta. modulators; anti-CDK compounds; HDAC inhibitors; and the like.

In some embodiments, the methods further comprise administering one or more additional therapeutic agents that bind to and inhibit anti-apoptotic proteins (e.g., agents that inhibit anti-apoptotic BCL-2 proteins), such as ABT-263, obatoclax, gossypol derivatives, IAP inhibitors, and stapled peptides that target anti-apoptotic proteins (e.g., MCL-1 SAHB, BID SAHB, BAD SAHB, BIM SAHB, and the like).

In some embodiments, the methods further comprise administering one or more additional therapeutic agents (e.g., pro-apoptotic agents) that bind to and activate the pro-apoptotic activity of BAX (e.g., BIM SAHB_{A2}). Additional examples of compounds which bind to and activate the pro-apoptotic activity of BAX may be found, for example, in U.S. Pat. No. 9,303,024; U.S. Patent Publication No. US 2016-0171150; Gavathiotis et al, Nat. Chem. Biol. 2012, 8:639-645; Brahmbhatt et al, Biochem. J. 2016, 473:1073-1083; Xin et al, Nat. Commun. 2014, 5:4935; and Zhao et al, Mol. Cell. Biol. 2014, 34:1198-1207; the disclosures of each of which are incorporated herein by reference in their entireties.

In some embodiments, the methods further comprise administering one or more additional therapeutic agents that induce or stimulate apoptosis. Agents that induce apoptosis include, but are not limited to, radiation (e.g., X-rays, gamma rays, UV); kinase inhibitors (e.g., Epidermal Growth Factor Receptor (EGFR) kinase inhibitor, Vascular Growth Factor Receptor (VGFR) kinase inhibitor, Fibroblast Growth Factor Receptor (FGFR) kinase inhibitor, Platelet-derived Growth Factor Receptor (PDGFR) kinase inhibitor, and Bcr-Abl kinase inhibitors such as GLEEVEC); antisense molecules; antibodies (e.g., HERCEPTIN, RITUXAN, ZEVALIN, and AVASTIN); anti-estrogens (e.g., raloxifene and tamoxifen); anti-androgens (e.g., flutamide, bicalutamide, finasteride, aminoglutethamide, ketoconazole, and corticosteroids); cyclooxygenase 2 (COX-2) inhibitors (e.g., celecoxib, meloxicam, NS-398, and non-steroidal anti-inflammatory drugs (NSAIDs)); anti-inflammatory drugs (e.g., butazolidin, DECADRON, DELTASONE, dexamethasone, dexamethasone intensol, DEXONE, HEXADROL, hydroxychloroquine, METICORTEN, ORADEXON, ORASONE, oxyphenbutazone, PEDIAPRED, phenylbutazone, PLAQUENIL, prednisolone, prednisone, PRELONE, and TANDEARIL); and cancer chemotherapeutic drugs (e.g., irinotecan (CAMPTOSAR), CPT-11, fludarabine (FLUDARA), dacarbazine (DTIC), dexamethasone, mitoxantrone, MYLOTARG, VP-16, cisplatin, carboplatin, oxaliplatin, 5-FU, doxorubicin, gemcitabine, bortezomib, gefitinib, bevacizumab, TAXOTERE, or TAXOL); cellular signaling molecules; ceramides and cytokines; and staurosporine, and the like.

In some embodiments, the subject is a subject in need thereof (e.g., a subject identified as being in need of such treatment, such as a subject having, or at risk of having, one or more of the diseases provided herein). Identifying a subject in need of such treatment can be in the judgment of a subject or a health care professional and can be subjective (e.g., opinion) or objective (e.g., measurable by a test or diagnostic method).

In some embodiments, the subject has not previously undergone chemotherapy. In some embodiments, the subject is not suffering from, or at risk of, thrombocytopenia, such as thrombocytopenia resulting from chemotherapy, radiation therapy, or bone marrow transplantation as treatment for cancer or lymphoma.

In some embodiments, the additional therapeutic agent is administered prior to, simultaneously with, or after administration of a composition provided herein. In some embodiments, the composition provided herein is administered during a surgical procedure. In some embodiments, the composition provided herein is administered in combination with an additional therapeutic agent during a surgical procedure.

As used herein, the term “treating” or “treatment” refers to one or more of (1) inhibiting the disease; for example, inhibiting a disease, condition or disorder in an individual who is experiencing or displaying the pathology or symptomatology of the disease, condition or disorder (i.e., arresting further development of the pathology and/or symptomatology); and (2) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease or reducing or alleviating one or more symptoms of the disease. In some embodiments, such terms refer to one, two, three or more results following the administration of one or more therapies: (1) a stabilization, reduction or elimination of a cancer cell population, (2) an increase in the length of cancer remission, (3) a decrease in the recurrence rate of a cancer, (4) an increase in the time to recurrence of a cancer, and (6) an increase in the survival of the patient.

### EXAMPLES

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results.

### Example 1. Peptide Synthesis

Solid-state peptide synthesis using Fmoc chemistry was performed as previously described (see e.g., Bird et al, Methods Enzymol. 2008, 446:369-386; Bird et al, Curr. Protoc. Chem. Biol. 2011, 3:99-117). The vMIA (¹³¹EALKKALRRHRFLWQRRQRA¹⁵⁰-CONH₂) (SEQ ID NO:2) and BIM SAHB_{A2} (¹⁴⁵Ac-EIWIAQELRXIGDXFNAYYA¹⁶⁴-CONH₂, X=stapling amino acid) (SEQ ID NO:3) peptides were N-terminally derivatized with either an acetyl group or fluorescein isothiocyanate (FITC)-β-alanine for the indicated applications in NMR and biochemical experiments. Peptides were purified by LC-MS to >95% purity and quantified by amino acid analysis. Lyophilized peptides were reconstituted in 100% DMSO or DMSO-d₆ and diluted into the indicated aqueous buffers for experimental use.

### Example 2. Expression and Purification of Full-Length BAX

Recombinant, full-length BAX was expressed in BL21 (DE3) E. coli using the pTYB1 vector (see e.g., Suzuki et al, Cell, 2000, 103:645-654; Gavathiotis et al, Nature, 2008, 455:1076-1081). Cell pellets were resuspended in 20 mM Tris, 250 mM NaCl, pH 7.2 and lysed by two passes through a microfluidizer (Microfluidics) chilled to 4° C. The lysate was clarified by centrifugation at 20,000 rpm. BAX was purified by batch affinity binding at 4° C. using chitin resin (New England Biolabs), followed by loading onto gravity flow columns for washing and elution. The intein-chitin binding domain tag was cleaved by 36-hour incubation in 50 mM dithiothreitol at 4° C. Pure protein was isolated by size exclusion chromatography (Superdex 75 10/300; 20 mM potassium phosphate, pH 6.2) using an FPLC system (GE Healthcare Life Sciences).

### Example 3. Fragment Screening by STD-NMR

The Ro3 diversity compound library was purchased from Maybridge, characterized by ¹H-NMR, and then pooled in groups of 10 to minimize spectral overlap. Forty compounds were excluded prior to screening as part of a quality control measure that identifies poorly-behaved compounds. Fragment pools were added to a 5 μM solution of unlabeled, full-length human BAX in 20 mM potassium phosphate buffer, pH 6.2 in 10% (v/v) D₂O, resulting in a final compound concentration of 300 μM. The mixing and loading of samples into a 5-mm NMR tube was performed using a liquid handling robot (Gilson). STD-NMR measurements were acquired at 25° C. on a Varian Inova 500-MHz spectrometer equipped with a helium-cooled cryoprobe. Low power saturation of the protein was achieved with a series of 50 ms Gaussian pulses for a total of 3 seconds; on-resonance irradiation was performed at 0.8 ppm, and off-resonance irradiation at 30 ppm. Standard excitation sculpting was used for solvent suppression. Each experiment was run for 14 min. The results were initially analyzed by comparing the on and off resonance STD spectra for each pool to determine the presence of binders, with 37 out of 96 pools demonstrating evidence of protein interaction. Subsequently, each pool was analyzed to identify individual binders using inhouse display analysis and display software, which allowed for precise alignment of on- and off-resonance spectra. Compounds in pools that yielded a positive STD signal were then subdivided into groups of three for retesting. Those compounds that exhibited STD in both experiments were reordered from Maybridge and tested both as single compounds and in competitive binding experiments.

To generate recombinant, full-length BAX of sufficient quantity and stability to execute a ligand screen was obtained, the production method was scaled up to an overall culture volume of 48 liters, and sequential lysis of bacterial pellets was performed using a temperature-controlled microfluidizer (set at 4° C.), followed by batch binding of the lysate to chitin affinity resin, dithiothreitol (DTT) elution, and purification by size exclusion chromatography. Using this approach, 21.6 mg of BAX protein was generated at a concentration of 0.64 mg/mL for initial screening, representing an overall yield of 0.45 mg of pure, full length protein per liter of bacterial culture. Ligand screening by saturation transfer difference (STD) NMR was then used to identify molecules that interact with BAX, as described above. The STD-NMR measured the change in ¹H-NMR signal of a ligand following selective irradiation of the target protein, where transfer of magnetization from protein to ligand causes a decrease in signal that reflects ligand-protein interaction.

The Maybridge Ro3 library of 1000 compounds was used for the BAX screen. Of the 96 pools analyzed, a positive STD signal was detected in 37, which represented 86 individual hits that were then rescreened in pools of three, ultimately yielding 56 confirmed interactors (FIG. 1b). Fifty-three commercially available compounds were ordered, retested by STD as singletons, and confirmed as BAX-Interacting Fragments (BIFs 1-53). The results obtained from STD NMR and liposomal release assays are shown in Table 1. Structures of active compounds BIF-1 to BIF-53 are shown in Table 2.

TABLE 1

| | | STD-NMR competition | | | |
|---|---|---|---|---|---|
| | STD- | fragment/peptide | | Liposomal Release Assay | |
| | Binder | vMIA | BIM SAHB_{A2} | Sensitizer | Activator |
| BIF-1 | + | + | − | − | − |
| BIF-2 | + | − | + | − | − |
| BIF-3 | + | − | − | − | − |
| BIF-4 | + | − | − | − | − |
| BIF-5 | + | − | − | − | − |
| BIF-6 | + | + | − | − | − |
| BIF-7 | + | − | − | − | − |
| BIF-8 | + | − | − | − | − |
| BIF-9 | + | − | − | − | − |
| BIF-10 | + | − | − | − | − |
| BIF-11 | + | − | − | − | − |
| BIF-12 | + | − | − | − | − |
| BIF-13 | + | + | − | − | − |
| BIF-14 | + | − | − | − | − |
| BIF-15 | + | − | − | − | + |
| BIF-16 | + | − | − | − | − |
| BIF-17 | + | − | − | − | − |
| BIF-18 | + | − | − | − | − |
| BIF-19 | + | − | − | + | − |
| BIF-20 | + | − | + | − | − |
| BIF-21 | + | − | − | − | − |
| BIF-22 | + | − | − | − | − |
| BIF-23 | + | − | + | − | + |
| BIF-24 | + | − | − | − | − |
| BIF-25 | + | + | − | + | − |
| BIF-26 | + | + | − | − | − |
| BIF-27 | + | + | − | + | − |
| BIF-28 | + | − | − | − | + |
| BIF-29 | + | − | − | − | − |
| BIF-30 | + | − | − | − | − |
| BIF-31 | + | + | − | − | − |
| BIF-32 | + | − | − | − | − |
| BIF-33 | + | + | − | − | − |
| BIF-34 | + | − | − | − | − |
| BIF-35 | + | − | − | − | − |
| BIF-36 | + | − | − | − | − |
| BIF-37 | + | − | − | − | − |
| BIF-38 | + | − | − | − | − |
| BIF-39 | + | − | − | − | − |
| BIF-40 | + | − | − | − | − |
| BIF-41 | + | − | − | − | − |
| BIF-42 | + | − | − | − | − |
| BIF-43 | + | − | − | − | − |
| BIF-44 | + | + | − | + | − |
| BIF-45 | + | − | + | + | − |
| BIF-46 | + | − | + | + | − |
| BIF-47 | + | − | − | − | − |
| BIF-48 | + | − | − | − | − |
| BIF-49 | + | + | − | − | + |
| BIF-50 | + | + | − | − | − |
| BIF-51 | + | − | − | + | − |
| BIF-52 | + | − | − | − | − |
| BIF-53 | + | − | − | − | − |
| | 53 | 11 | 5 | 8 | 4 |

TABLE 2

| | |
|---|---|
| 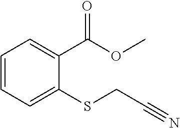 | BIF-1 |
| 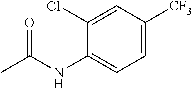 | BIF-2 |
| 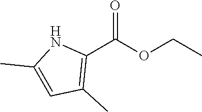 | BIF-3 |
| 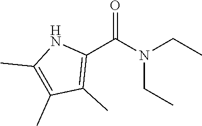 | BIF-4 |
| 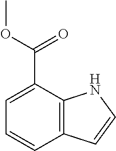 | BIF-5 |
| 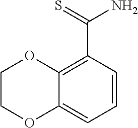 [SMILES: NC(=S)c1cccc2c1OCCO2] | BIF-6 |
| 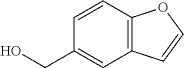 [SMILES: OCc1ccc2occc2c1] | BIF-7 |
| 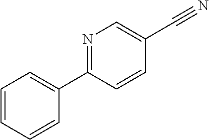 [SMILES: N#Cc1ccc(-c2ccccc2)nc1] | BIF-8 |
| 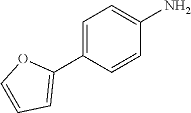 [SMILES: Nc1ccc(-c2ccco2)cc1] | BIF-9 |
| 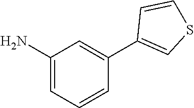 [SMILES: Nc1cccc(-c2ccsc2)c1] | BIF-10 |
| 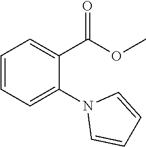 | BIF-11 |
| 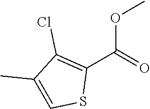 | BIF-12 |
| 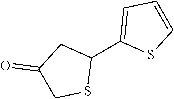 [SMILES: O=C1CSC(c2cccs2)C1] | BIF-13 |
| 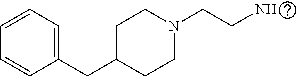 [SMILES: NCCN1CCC(Cc2ccccc2)CC1] | BIF-14 |
| 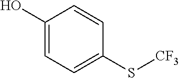 [SMILES: Oc1ccc(SC(F)(F)F)cc1] | BIF-15 |
| 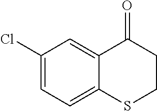 [SMILES: O=C1CCSc2ccc(Cl)cc21] | BIF-16 |
| 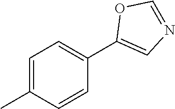 | BIF-17 |
| 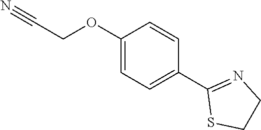 [SMILES: N#CCOc1ccc(C2=NCCS2)cc1] | BIF-18 |
| 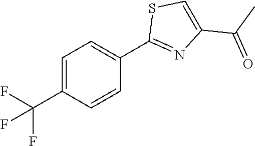 [SMILES: CC(=O)c1csc(-c2ccc(C(F)(F)F)cc2)n1] | BIF-19 |
| 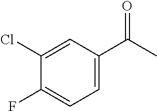 | BIF-20 |
| 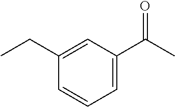 | BIF-21 |
| 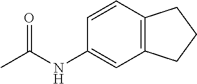 | BIF-22 |
| 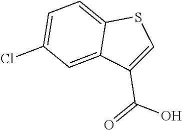 [SMILES: O=C(O)c1csc2ccc(Cl)cc12] | BIF-23 |
| 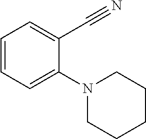 [SMILES: N#Cc1ccccc1N1CCCCC1] | BIF-24 |
| 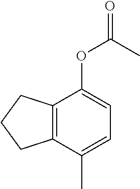 | BIF-25 |
| 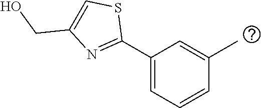 | BIF-26 |
| 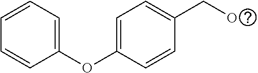 [SMILES: OCc1ccc(Oc2ccccc2)cc1] | BIF-27 |
| 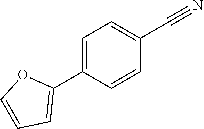 [SMILES: N#Cc1ccc(-c2ccco2)cc1] | BIF-28 |
| 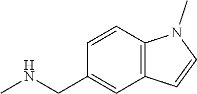 | BIF-29 |
| 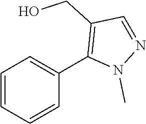 | BIF-30 |
| 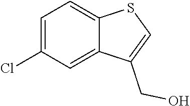 [SMILES: OCc1csc2ccc(Cl)cc12] | BIF-31 |
| 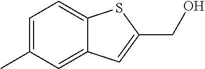 | BIF-32 |
| 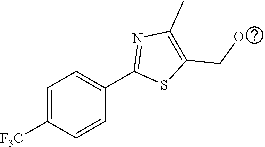 [SMILES: Cc1nc(-c2ccc(C(F)(F)F)cc2)sc1CO] | BIF-33 |
| 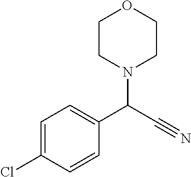 [SMILES: N#CC(c1ccc(Cl)cc1)N1CCOCC1] | BIF-34 |
| 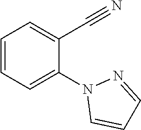 [SMILES: N#Cc1ccccc1-n1cccn1] | BIF-35 |
| 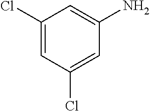 | BIF-36 |
| 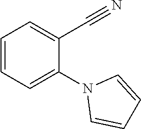 [SMILES: N#Cc1ccccc1-n1cccc1] | BIF-37 |
| 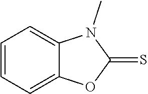 | BIF-38 |
| 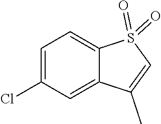 | BIF-39 |
| 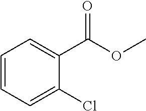 | BIF-40 |
| 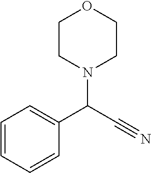 [SMILES: N#CC(c1ccccc1)N1CCOCC1] | BIF-41 |
| 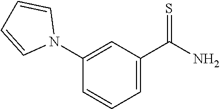 [SMILES: NC(=S)c1cccc(-n2cccc2)c1] | BIF-42 |
| 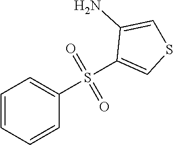 [SMILES: Nc1cscc1S(=O)(=O)c1ccccc1] | BIF-43 |
| 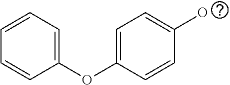 [SMILES: Oc1ccc(Oc2ccccc2)cc1] | BIF-44 |
| 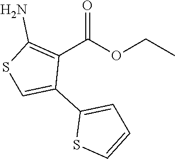 [SMILES: CCOC(=O)c1c(-c2cccs2)csc1N] | BIF-45 |
| 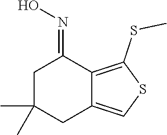 | BIF-46 |
| 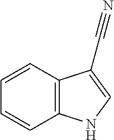 [SMILES: N#Cc1c[nH]c2ccccc12] | BIF-47 |
| 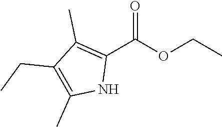 | BIF-48 |
| 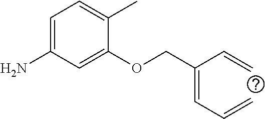 [SMILES: C=C/C=C(\C=C)COc1cc(N)ccc1C] | BIF-49 |
| 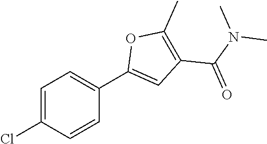 [SMILES: Cc1oc(-c2ccc(Cl)cc2)cc1C(=O)N(C)C] | BIF-50 |
| 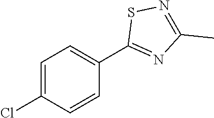 | BIF-51 |
| 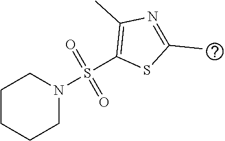 [SMILES: Cc1nc(C)c(S(=O)(=O)N2CCCCC2)s1] | BIF-52 |
| 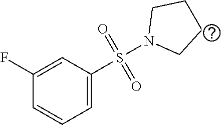 [SMILES: O=S(=O)(c1cccc(F)c1)N1CCCC1] | BIF-53 |

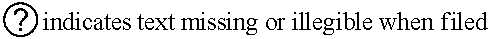

### Example 4. Liposomal Release Assay

Large unilamellar vesicles (LUVs) with a lipid composition similar to the outer mitochondrial membrane were formed by liposome extrusion as previously described (see e.g., Leshchiner et al, Proc. Natl. Acad. Sci. U.S.A., 2013, 110:E986-995; Lovell et al, Cell, 2008, 135:1074-1084). Briefly, a lipid mixture containing a 48:28:10:10:4 molar ratio of phosphatidylcholine, phosphatidylethanolamine, phosphatidylinositol, dioleoyl phosphatidylserine, and tetraoleolyl cardiolipin (Avanti Polar Lipids) was generated in chloroform. Lipid films were formed by evaporation of solvent, initially under nitrogen gas and then by overnight vacuum, followed by storage at −80° C. under nitrogen. Lipid films were hydrated in 1 mL assay buffer (10 mM HEPES, 200 mM KCl, 1 mM MgCl₂, pH 7.0) and mixed with the fluorophore and quencher pair, 8-aminonaphthalene-1,3,6-trisulfonic acid (ANTS, 12.5 mM) and p-xylene-bis-pyridinium bromide (DPX, 45 mM). Liposomes were formed by 5 freeze/thaw cycles followed by extrusion through a 100 nm polycarbonate membrane and purified using a Sepharose CL-2B size exclusion column. For measurement of BAX activation, BAX (750 nM) was added to the indicated concentration of molecular fragment in the presence of liposomes, followed by BIM SAHBA2 (750 nM), at the indicated time points. The assay was carried out in black opaque 384 well plates (30 μl per well). ANTS/DPX release was monitored over time at room temperature in a spectrofluorometer (Tecan Infinite M1000) using an excitation wavelength of 355 nm, an emission wavelength of 540 nm, and a bandwidth of 20 nm. Maximal release was determined by the addition of Triton X-100 to a final concentration of 0.2% (v/v). Percent release was calculated according to Equation 1 shown below, where F is the observed release, and F₀ and F₁₀₀ are baseline and maximal fluorescence, respectively.

((F−F₀)/(F₁₀₀−F₀))×100 Equation 1.

To determine if any of the identified BIFs influenced the function of BAX, the 53 BIFs were screen in the liposomal release assay described above, designed to identify both (1) direct BAX activators and (2) sensitizers or inhibitors of direct BAX activation induced by a stapled BIM BH3 helix, BIM SAHB_{A2} (aa 145-164) (see e.g., Gavathiotis et al, Nature, 2008, 455:1076-1081). First, baseline fluorescence with liposomes and compound alone was read, followed by the addition of BAX to evaluate for direct activation; then, BIM SAHB_{A2} was added to this mixture and the effect of the combination monitored, and compared with the triggering activity of BIM SAHB_{A2} and BAX in the absence of compound. Using this assay format, 4 direct activators of BAX-mediated liposomal release and 8 sensitizers of BIM SAHB_{A2}-triggered, BAX activation were identified, as shown in Table 1. The direct activator profile was exemplified by the positive control BIM SAHB_{A2} peptide, which induced time-responsive liposomal release in the presence of BAX alone (FIG. 1c). A novel sensitizer profile was most strikingly reflected by the activity of BIF-44, which had a minimal effect on BAX when incubated as a single agent, but when combined with BIM SAHB_{A2}, the maximal BAX-mediated release jumped from 50% with BIM SAHB_{A2} alone to 80% for the combination, and displayed more rapid kinetics (FIG. 1c).

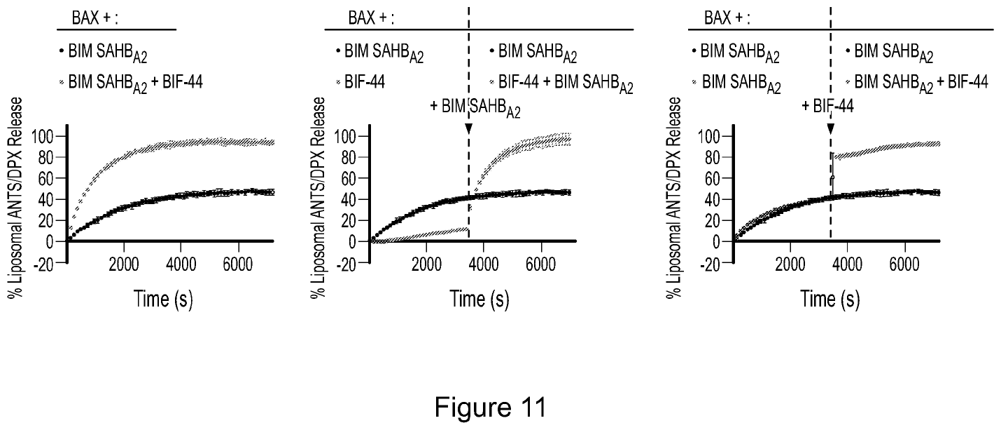

In addition, it was found that BIF-44 sensitization of BAX-mediated liposome release was independent of the order of addition of BIF-44 and BIM SAHB_{A2}. The same level of BAX activation was achieved whether BIF-44 was added simultaneously (left), before (right), or after (middle) the addition of BIM SAHB_{A2}, as shown in FIG. 11.

### Example 5. Competition STD-NMR

Individual compounds were added to 5 μM BAX with or without 5 μM competitor peptide in 20 mM potassium phosphate buffer, pH 6.2. STD-NMR was measured as described above. Fragments that were competed by vMIA or BIM SAHB_{A2} showed a decreased saturation-transfer difference in the presence of peptide relative to no peptide.

In prior work characterizing direct BAX activator molecules (i.e., BAMs), direct competition between BAMs and BIM SAHB_{A2} was observed at the BH3-trigger site (see e.g., Gavathiotis et al, Nat. Chem. Biol. 2012, 8:639-645). In evaluating the newly-identified BAX-sensitization activity, it was surprisingly found that BIM SAHB_{A2} had no effect on the STD signal (FIG. 1d), raising the possibility of an alternative interaction mechanism for BIF-44.

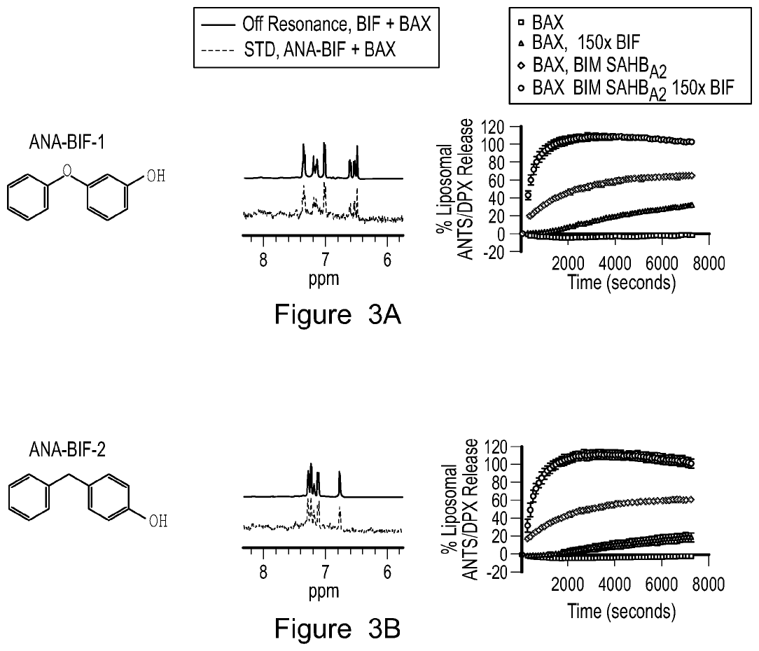

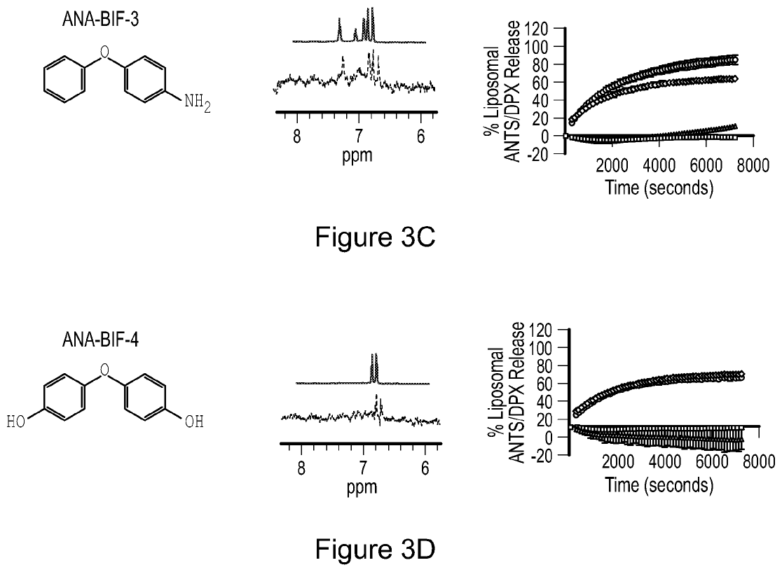

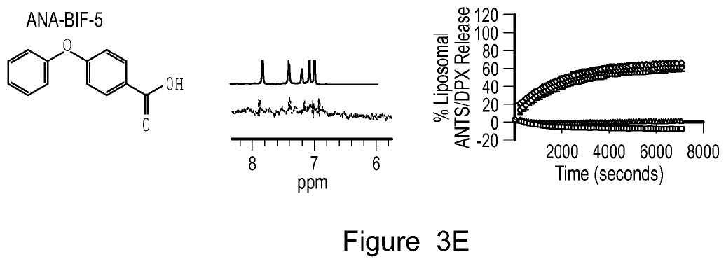

To evaluate the structure-based reproducibility and selectivity of the observed BIF-44 activity, the binding and functional properties of a series of BIF-44 analogs were evaluated. It was found that BIF-44-like diaryl ethers that either replace the hydroxyl group with an amine in the same position, shift the hydroxyl to the meta position, or replace the ether linkage with a methylene group, all retain BAX-binding activity that is competed by vMIA, as assessed by STD NMR, and demonstrated robust BAX-sensitization activity (FIGS. 3a-3c). In contrast, diaryl ethers that bear a para-hydroxyl group in the second aromatic ring or that replace the BIF-44 hydroxyl with a carboxylate group, showed little to no BAX-binding or sensitization activity (FIGS. 3d-3e). These data provided evidence for a structure activity relationship that supports the specificity of action of BIF-44 in binding to BAX, competing with vMIA, and sensitizing BH3-mediated BAX activation.

### Example 6. CPMG NMR

CPMG experiments were performed using standard methods (see e.g., Hajduk et al, J. Am. Chem. Soc., 1997, 119:12257-12261). NMR analyses employed BIF-44 at a concentration of 300 μM, with or without added BAX (5 μM), in a 20 mM potassium phosphate buffer, pH 6.2. A 0.5 millisecond tau delay (1 ms per CPMG echo cycle) was applied, with the number of echo cycles corresponding to 500 ms. Excitation sculpting was used for solvent suppression, as reported (see e.g., Hwang et al, J. Magn. Reson. A, 1995, 112:275-279).

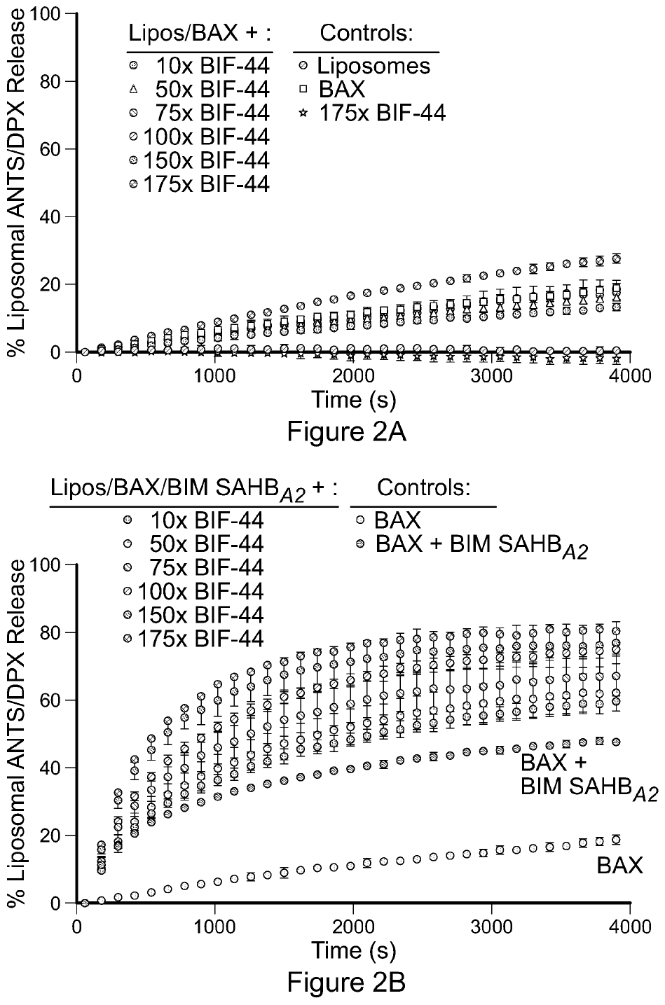

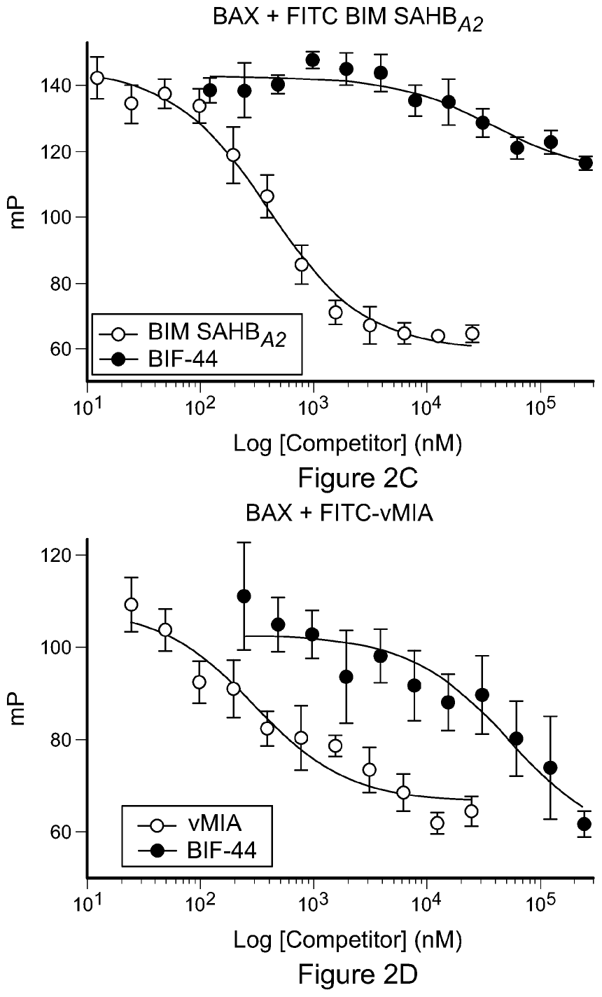

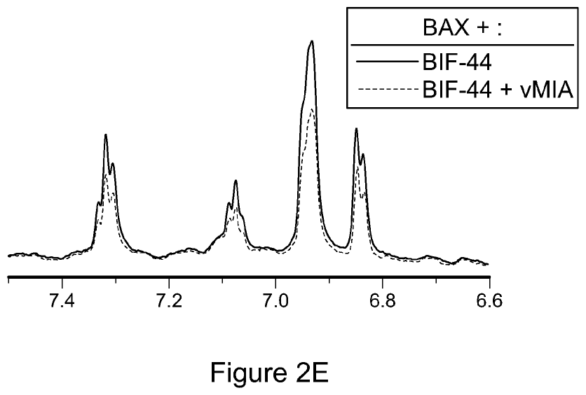

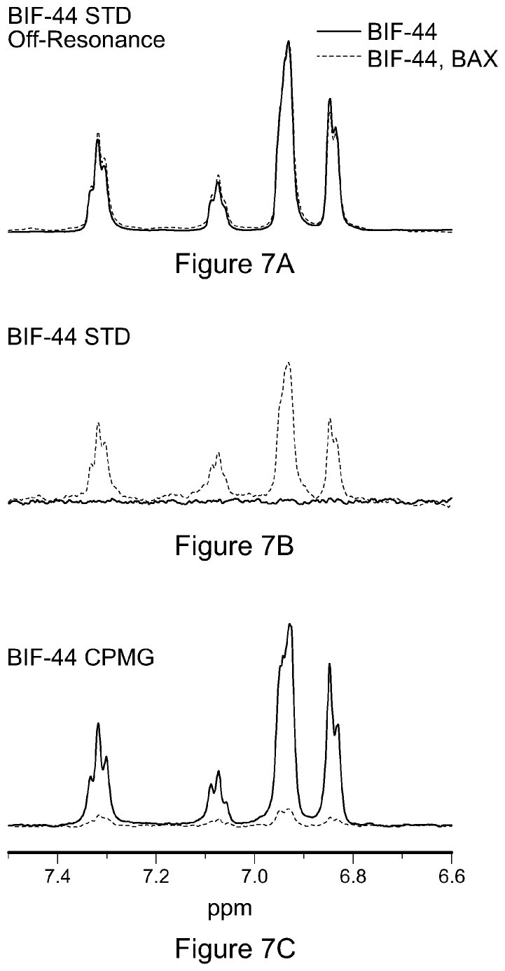

Given the results obtained for BIF-44 in both the liposomal release and BIM SAHB_{A2}-competitive STD secondary screens, the BIF-44/BAX interaction findings based on STD were corroborated (FIGS. 7a-7b) using an orthogonal NMR measure. Carr-Purcell-Meiboom-Gill (CPMG)-NMR was applied as described above, a method that takes advantage of the faster T2 relaxation time of protein compared to ligand, to monitor for a potential change in BIF-44 signal upon incubation with BAX. The formation of a protein-ligand complex reduces the relaxation time of the ligand, resulting in a measurable decrease in 1H-NMR signal (see e.g., Dias et al, ACS Med. Chem. Lett. 2014, 5:23-28; Stockman et al, Prog. Nucl. Mag. Reson. Spectrosc. 2002, 41:187-231). In the presence of BAX, a sharp reduction in signal was observed, indicative of BIF-44 binding (FIG. 7c). In addition, it was confirmed that BIF-44 had little to no independent triggering effect on BAX-mediated liposomal release when applied using a broad 10-175:1 molar ratio of BIF-44 to BAX (FIG. 2a), but in the presence of BIM SAHB_{A2}, BIF-44 dose-responsively enhanced both the kinetics and maximum level of BAX mediated liposomal release (FIG. 2b).

### Example 7. Fluorescence Polarization (FP) Assay

FITC-peptide (25 nM) was incubated with a serial dilution of recombinant, full length BAX in binding buffer (20 mM Potassium phosphate, pH 6.2). For competitive FP, FITC-peptide (25 nM) was mixed with a fixed concentration of BAX (250 nM) and incubated with a serial dilution of acetylated peptide or a compound described herein. Fluorescence polarization was measured at equilibrium using a SpectraMax M5 microplate reader. Nonlinear regression analysis of dose-response curves was performed using Prism software 7 (GraphPad).

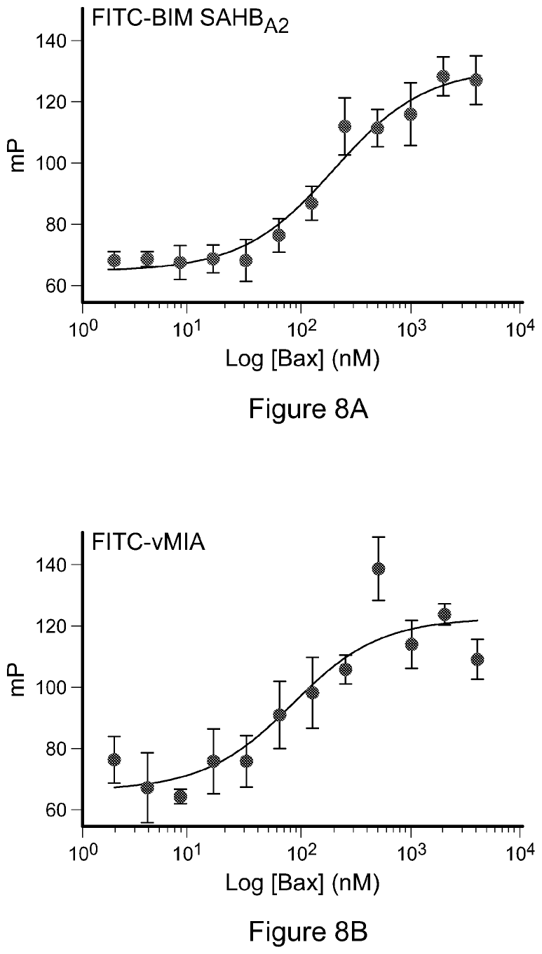

Finally, the absence of BIM SAHB_{A2} competition for BIF-44 engagement of BAX was confirmed, as initially demonstrated by STD (FIG. 1d), using the alternative method of competitive fluorescence polarization assay. For this experiment, the direct interaction between FITC-BIM SAHB_{A2} and BAX was employed (FIG. 8a) as the basis for comparative competition by N-terminal acetylated BIM SAHB_{A2} and BIF-44. Whereas Ac-BIM SAHB_{A2} dose-responsively competed with FITC-BIM SAHB_{A2} for BAX binding, BIF-44 had little to no effect (FIG. 2c). Thus, in a series of tertiary screening experiments it was determined that BIF-44 directly binds to BAX, an interaction not competed by BIM SAHB_{A2}, and dose-responsively sensitizes BIM SAHB_{A2}-triggered, BAX-mediated membrane poration.

It was also tested whether the identified BIFs could compete with the inhibitory vMIA peptide for BAX interaction. vMIA is a cytomegalovirus protein implicated in blocking BAX-mediated apoptosis, which ensures host cell survival during viral infection and replication (see e.g., Amoult et al, Proc. Natl. Acad. Sci. U.S.A. 2004, 101:7988-7993; Poncet et al, J. Biol. Chem. 2004, 279:22605-22614). The BAX-binding domain of vMIA achieves its inhibitory effect by binding to a discrete pocket formed by the flexible loops between helices α1/α2, α3/α4, and α5/α6 and a portion of the C-terminal α9 helix, preventing BAX-activating conformational changes by stabilizing the α3/α4 and α5/α6 hairpins8. Surprisingly, BIF-44 dose-responsively competed with FITC-vMIA for BAX interaction, as assessed by competitive fluorescence polarization assay (FPA) (FIG. 2d, FIG. 8b). This finding was confirmed by competitive-STD NMR, which demonstrated a reduction in the BIF-44 STD signal upon co-incubation with vMIA peptide (FIG. 2e).

### Example 8. HSQC NMR

Uniformly ¹⁵N-labeled recombinant BAX was generated as previously described (see e.g., Suzuki et al, Cell, 2000, 103:645-654; Gavathiotis et al, Nature, 2008, 455:1076-1081). Protein samples with the indicated molar ratio of fragment were prepared in 25 mM sodium phosphate, 50 mM NaCl solution at pH 6.0 in 10% (v/v) D₂O. Correlation ¹H-¹⁵N HSQC spectra were acquired at 25° C. on a Bruker 600 MHz NMR spectrometer equipped with a cryogenic probe, processed in Topspin (Bruker) and analyzed using CcpNmr Analysis (see e.g., Vranken et al, Proteins, 2005, 59:687-696). The weighted average chemical shift difference was calculated as Equation 2, where ΔH/ΔN is the change in p.p.m. of ¹H or ¹⁵N for the indicated crosspeak.

$$\begin{matrix} Δ = \text{?} \sqrt{1 \text{/} 2 \text{?} ( ( Δ H ) \text{?} + ( Δ N \text{/} \text{?} ) \text{?} )} & Equation 2 \\ \text{?} \text{indicates text missing or illegible when filed} & \end{matrix}$$

The absence of a bar indicated no chemical shift difference, or the presence of a proline or residue that was overlapped or not assigned. BAX cross-peak assignments were applied as previously reported (see e.g., Suzuki et al, Cell, 2000, 103:645-654). The significance threshold for the chemical shift changes was calculated based on the average chemical shift across all residues plus the standard deviation, in accordance with standard methods (see e.g., Marintchev et al, Methods Enzymol, 2007, 430:283-331).

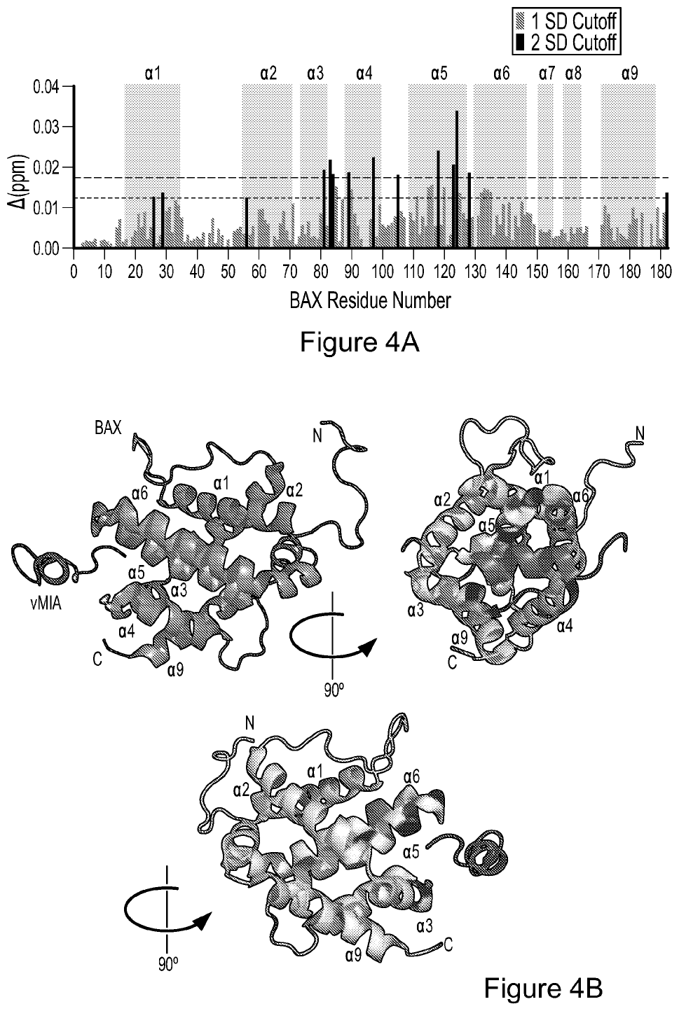

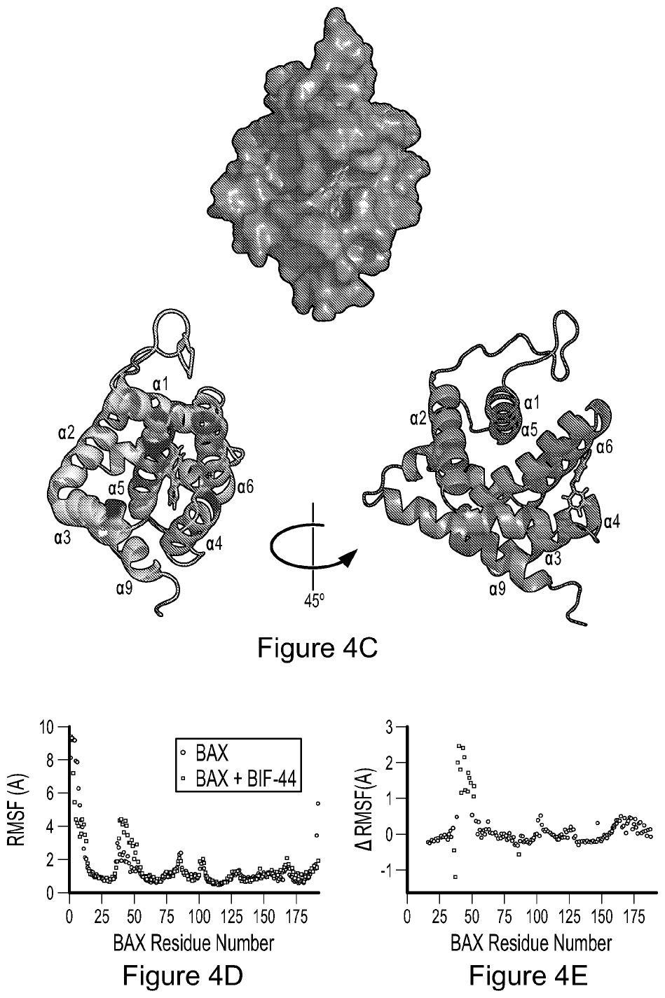

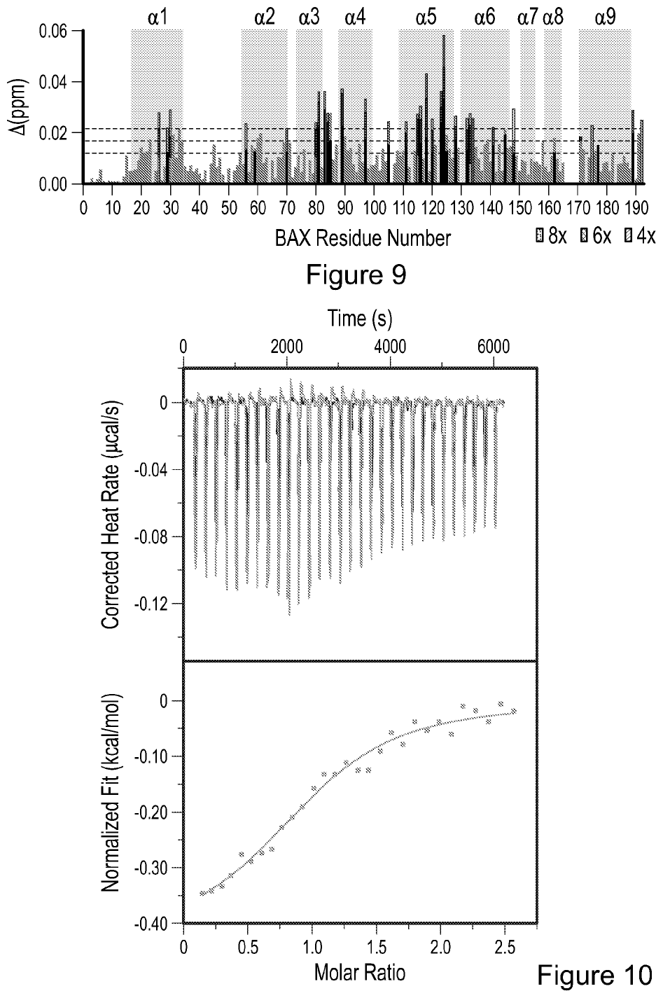

¹⁵N-BAX NMR was performed upon BIF-44 titration, and a series of focal, dose-responsive chemical shift changes were identified that colocalized to the very region implicated in the vMIA binding site on BAX (FIG. 4a, FIG. 9). The most prominent changes (2 SD) localized to the junction of the α3-α4 and α5-α6 hairpins, which juxtapose to form a binding interface (FIG. 4b). Especially intriguing were more subtle changes (1 SD) that become amplified with increasing BIF-44 dosage and localized both to the internal helical regions of α5 and α6 (i.e., BAX's hydrophobic core), and the neighboring internal interaction surfaces between α1 and α2 (FIGS. 4a-4b and FIG. 9), the latter helix being the critical BH3 motif that must become everted and exposed for BAX activation and oligomerization to ensue. Thus, these NMR data not only corroborated the STD and FPA data with respect to BIF-44 competition with vMIA at a strikingly similar interaction site, but also suggested that BIF-44 engagement induces structural reverberations transmitted through the α5-α6 hydrophobic core to the internal surfaces of α1 and α2, a region implicated in BIM BH3-mediated direct activation of BAX at its N-terminal surface (see e.g., Gavathiotis et al, Mol. Cell, 2010, 40:481-492; Gavathiotis et al, Nature, 2008, 455:1076-1081). To further develop a mechanistic hypothesis for the sensitization activity of BIF-44, the HSQC NMR results were applied to calculate a docked structure of the BIF-44/BAX complex. BIF-44 was shown engaging a deep pocket formed by the core hydrophobic α5 and α6 helices and the loop between α3 and α4 (FIG. 4c).

### Example 9. Molecular Docking

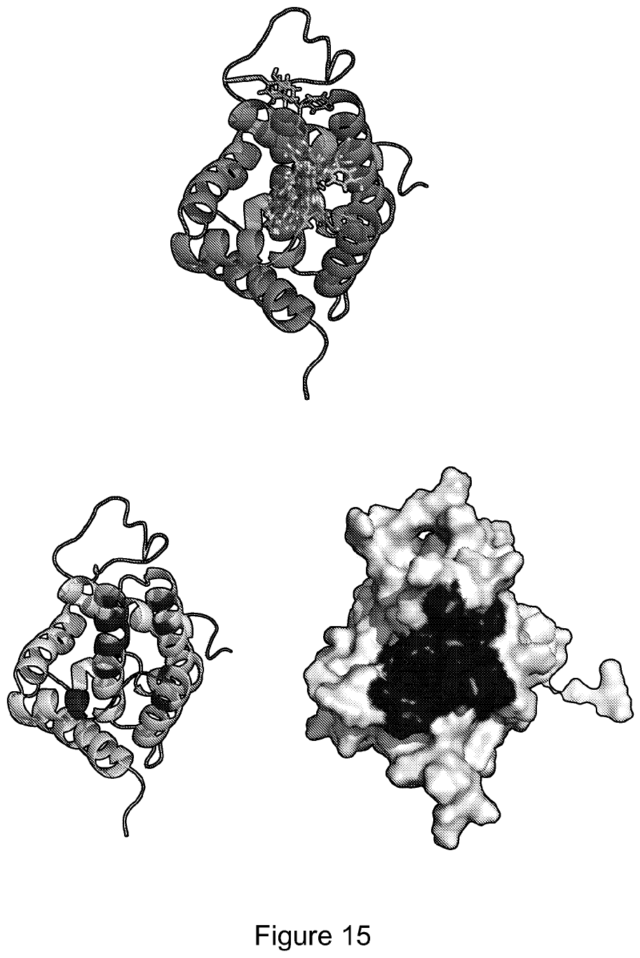

Molecular dynamics (MD) simulations were then performed that assessed protein movements in the presence or absence of BIF-44 at the docked site. The calculations suggested a specific increase in conformational flexibility involving the α1-α2 region of BAX (FIGS. 4d-4e), a site that is distant from the BIF-44 docking location but subject to allosteric sensing, as evidenced by the dose-responsive HSQC NMR results (FIG. 9). The novel binding site of BAX identified in the measurements described herein is shown in FIG. 15.

The Schrodinger software suite (Version 2016-2) was used for docking calculations. Conformations of molecule BIF-44 were generated in MacroModel using the OPLS3 forcefield (see e.g., Harder et al, J. Chem. Theory Comput. 2016, 12:281-296). Each of the 20 NMR conformations of Bax (PDB:1F16) was separately prepared using the default parameters in the PrepWiz wizard in Maestro. The docking receptor grid (radius 1 nm) was defined at the center of Ala124, the amino acid with the greatest HSQC shift. BIF-44 was then docked onto all 20 structures using Glide Extra Precision (XP) mode (see e.g., Friesner et al, J. Med. Chem. 2006, 49:6177-6196). The top-scoring poses were then manually inspected for consistency with experimentally-determined HSQC shifts for the complex.

### Example 10. Molecular Dynamics Simulation

The first NMR structure of BAX from PDB ID 1F16 was used as the starting structure for MD calculations. The protein was prepared using the default parameters of the Protein Preparation Workflow in Maestro (see e.g., Sastry et al, J. Comput. Aided Mol. Des. 2013, 27:221-234). Protonation states were those predicted to occur at pH 7.0 using the Epik module (see e.g., Shelley et al, J. Comput. Aided Mol. Des. 2007, 21:681-691). Protein was pre-soaked in a cubic box of TIP3P water molecules using the System Builder workflow in Desmond (see e.g., Jorgensen et al, The Journal of Chemical Physics, 1983, 79:926-935). The box was sized such that all peptide atoms were at least 1 nm from the boundaries. All overlapping solvent molecules were removed, the system was charge neutralized with appropriate counterions, and 150 mM NaCl was added to simulate buffer conditions. All MD simulations were performed using the Desmond package, with the OPLS3 forcefield applied to model all interactions. Periodic boundary conditions were maintained throughout. Long-range electrostatic interactions were calculated using the particle-mesh Ewald method (see e.g., Essmann et al, J. Chem. Phys. 1995, 103:8577-8593), and van der Waals and short-range electrostatic interactions were smoothly truncated at 0.9 nm. Constant system temperature of 300 K was maintained using Nose-Hoover thermostats (see e.g., Hoover et al, Phys. Rev. A. Gen. Phys. 1985, 31:1695-1697), and system pressure was maintained at 1 atm using the Martina-Tobias-Klein method (see e.g., Martyna et al, J. Chem. Phys. 1994, 101:4177-4189). The equations of motion were integrated using the RESPA integrator (see e.g., Humphreys et al, J Phys. Chem., 1994, 98:6885-6892), with a 2.0 fs timestep for bonded and short-range interactions and a 6.0 fs timestep for non-bonded interactions beyond the 0.9 nm cutoff. The default parameters in Desmond were used to relax the system prior to simulation (see e.g., Guo et al, Chem. Biol. Drug Des. 2010, 75:348-359). Following this procedure, a 100 ns production simulation was run and configurations saved at 4 ps intervals. All simulations were judged to have converged on the basis of radius of gyration calculations and RMSD.

### Example 11. Hydrogen-Deuterium Exchange Mass Spectrometry

Hydrogen-deuterium exchange mass spectrometry (HXMS) experiments were performed as previously described (see e.g., Barclay et al, Mol. Cell, 2015, 57:873-886; Lee et al, Nat. Struct. Mol. Biol. 2016, 23:600-607). Deuterium labeling was initiated with an 18-fold dilution into D₂O buffer (10 mM HEPES, 200 mM KCl, 1 mM MgCl₂, pD 7.0) of a pre-equilibrated (15 min, room temperature) aliquot of each BAX protein, molecule, peptide, and/or antibody (BAX BH3, Abgent AP1302a; BAX 6A7, Santa Cruz Biotechnology sc-23959) mixture. At the indicated time points, the labeling reaction was quenched with the addition of an equal volume of quench buffer (0.8 M guanidinium chloride, 0.8% formic acid [v/v]). Each deuterium labeling experiment was performed in at least duplicate. Proteolysis was performed by incubation on ice with 40 μg pepsin and 20 μg factor XIII for 5 min. Digested samples were then processed and analyzed as described previously (see e.g., Barclay et al, Mol. Cell, 2015, 57:873-886). The relative deuterium levels of identified peptides common to all evaluated conditions are shown. The error of determining the average deuterium incorporation for each peptide was at or below +/−0.25 Da. Relative deuterium levels for each peptide were calculated by subtracting the average mass of the undeuterated control sample from that of the deuterium-labeled sample. All mass spectra were processed using DynamX 3.0 (Waters Corporation). Deuterium levels were not corrected for back exchange and thus reported as relative (see e.g., Wales et al, Mass Spectrom. Rev. 2006, 25:158-170).

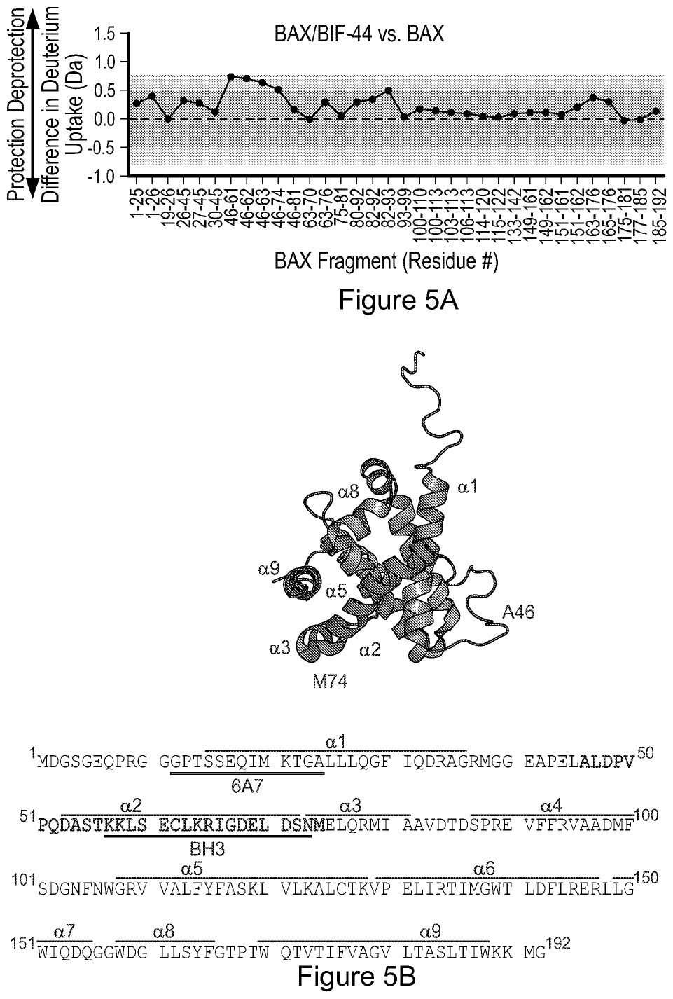

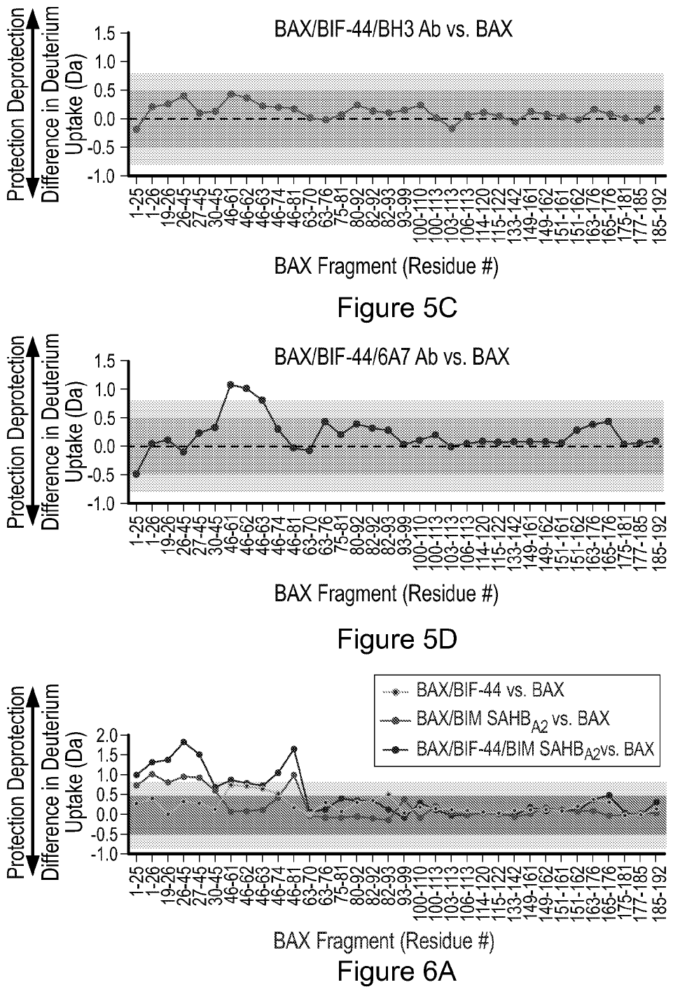

To evaluate whether the BIF-44 sensitization mechanism derived from allosteric mobilization of the α1-α2 region, which is implicated in BH3-mediated initiation of BAX activation via an N-terminal trigger site, comparative hydrogen-deuterium exchange mass (HXMS) spectrometry was performed as described above on a mixture of BAX and liposomes in the presence or absence of BIF-44. HXMS probes protein structure by measuring the deuterium incorporation of backbone amide hydrogens (see e.g., Engen et al, Anal. Chem. 2009, 81:7870-7875). When diluted into deuterium buffer, backbone hydrogens of flexible and/or exposed protein regions rapidly exchange with deuterium, whereas buried domains and/or those regions that contain hydrogen-bonding involving backbone amide hydrogens (such as in α-helices) demonstrate slowed or suppressed deuterium exchange (see e.g., Laiken et al, Biochemistry, 1969, 8:519-526; Printz et al, Proc. Natl. Acad. Sci. U.S.A. 1972, 69:378-382; Shi et al, Anal. Chem. 2013, 85:11185-11188). Here, it was found that upon incubation with BAX, BIF-44 induced focal deprotection of peptide fragments corresponding to amino acids 46-74, the region that encompasses the distal portion of the α1-α2 loop and the BH3 α2 helix of BAX (FIGS. 5a-b). To further validate the specificity of this finding, the influence of two antibodies, which bind to discrete regions of BAX, was tested on the observed BIF-44-induced deprotection. It was reasoned that if BIF-44 was specifically mobilizing or exposing the BH3 region of BAX, a BAX-BH3-specific antibody would promptly bind and suppress access of this region to deuterium exchange. Conversely, an antibody such as 6A7 that binds to an alternate region of the protein, which becomes exposed upon BH3-triggered BAX activation (aa 12-24) (see e.g., Gavathiotis et al, Nature, 2008, 455:1076-1081; Hsu et al, J. Biol. Chem. 1997, 272:13829-13834), would serve as a negative control. Indeed, it was found that the BH3 Ab selectively suppressed the observed deuterium exchange promoted by BIF-44 in the BAX BH3 region (FIG. 5c), whereas the 6A7 antibody had no inhibitory effect on BIF-44 mediated-deprotection (FIG. 5d). Taken together, the NMR, MD, and HXMS results, were consistent in linking BIF-44 binding at a noncanonical interaction site to allosteric mobilization of the α1-α2 region, where BH3-induced conformational changes initiate BAX activation.

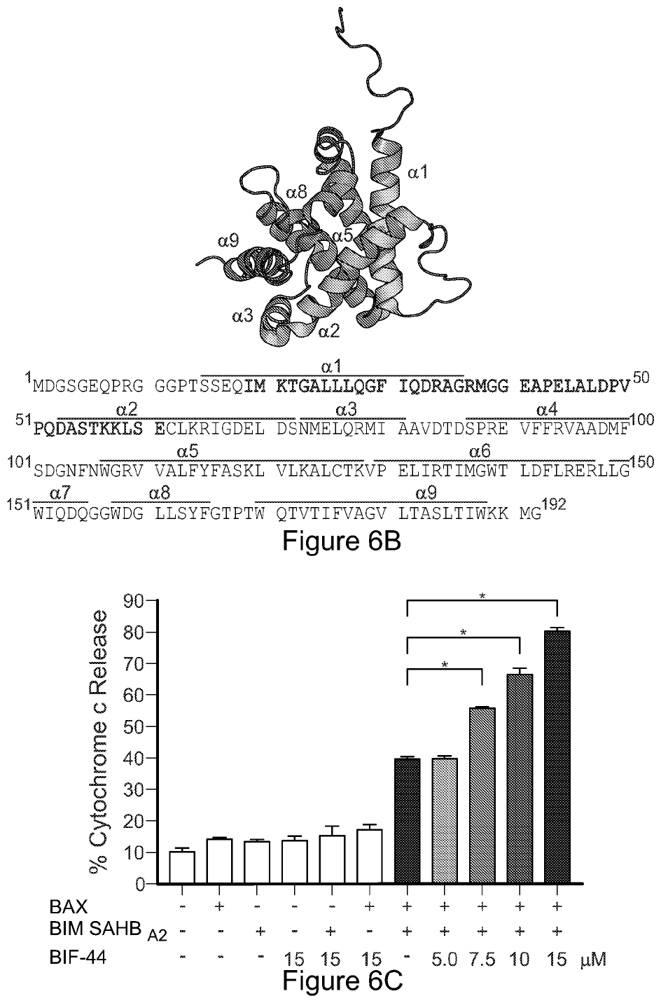

To examine how BIF-44 and BIM SAHB_{A2} engagement at distinct sites synergized to trigger BAX activation, HXMS analyses of BAX in the presence of BIF-44, BIM SAHB_{A2}, or the combination were performed. The hydrogen-deuterium exchange profiles of BIM SAHB_{A2} and BIF-44 were notably distinct, consistent with their different sites of engagement and distinct mechanisms of action. Whereas BIM SAHB_{A2} directly binds to the N-terminal trigger site formed by the confluence of α-helices 1 and 6 and displaces the α1-α2 loop leading to 6A7 epitope exposure (see e.g., Gavathiotis et al, Mol. Cell, 2010, 40:481-492; Barclay et al, Mol. Cell, 2015, 57:873-886). BIF-44 engaged a distant site, causing focal allosteric changes localized to the distal α1-α2 loop and the BH3 α2 helix (FIG. 6a). Combined treatment markedly amplified deprotection of essentially the entire α1-α2 region (FIGS. 6a-6b), consistent with the ability of BIF-44 to effectively sensitize BIM SAHB_{A2}-mediated conformational activation of BAX.

### Example 12. Mitochondrial Cytochrome c Release Assay

Liver mitochondria (0.5 mg/mL) from Alb^{Cre}Bax^{f/f}Bak^{-/-} mice were isolated and release assays performed as described (see e.g., Walensky et al, Mol. Cell, 2006, 24:199-210). Briefly, mitochondria were incubated with 100 nM BAX, 250 nM BIM SAHB_{A2} and/or the indicated concentrations of BIF-44 for 45 min at room temperature in experimental buffer (200 mM mannitol, 68 mM sucrose, 10 mM HEPES-KOH [pH 7.4], 110 mM KCl, 1 mM EDTA, protease inhibitor) (see e.g., Llambi et al, Mol. Cell, 2011, 44:517-531). The pellet and supernatant fractions were isolated by centrifugation, and cytochrome c was quantitated using a colorimetric ELISA (R&D Systems). Percent cytochrome c released into the supernatant (% cyto c release) was calculated according to Equation 3, where cyto cₛᵤₚ and cyto cₘₐₓ represent the amount of cytochrome c detected in the supernatant of compound- or 1% (v/v) Triton X-100-treated samples, respectively.

% cyto c release=[cyto cₛᵤₚ]/[cyto cₘₐₓ]*100 Equation 3.

Finally, to link these intriguing mechanistic findings to a physiologic context, the capacity of BIF-44 to sensitize BAX-mediated mitochondrial apoptosis, as measured by cytochrome c release from treated mouse liver mitochondria was tested.

Consistent with the synergy in conformational activation of the BAX N-terminal region, as observed by HXMS, BIF-44 dose-responsively sensitized BIM SAHB_{A2}-induced triggering of BAX-mediated cytochrome c release from mitochondria (FIG. 6c). Thus, the NMR screen identified a small molecule BAX sensitizer that facilitates the initiation of BH3-mediated direct BAX activation by a novel allosteric mechanism.

### Example 13. Isothermal Titration Calorimetry (ITC)

Binding affinity was measured by adding 0.15 mM recombinant BAX protein to the cell and injecting 2.0 μL of 1.0 mM ligand by syringe for a total of 30 injections using an Affinity ITC (TA instruments) at 25° C. BAX and BIF-44 solutions were prepared in 20 mM potassium phosphate buffer (pH 6.2), with a final concentration of 2% (v/v) DMSO. The samples were centrifuged for 15 min at 4° C. before titration. ITC experiments were performed in duplicate and the data analyzed with the NanoAnalyze software package (TA instruments) using a single binding site model and thermodynamic parameters calculated according to Equation 4, where ΔG, ΔH and ΔS are the changes in free energy, enthalpy and entropy of binding, respectively. Results of the ITC measurements are shown in FIG. 10.

ΔG=ΔH−TΔS=−RTlnK_{B} Equation 4.

### Example 14. NMR-Based Detection of Small Molecule Aggregators

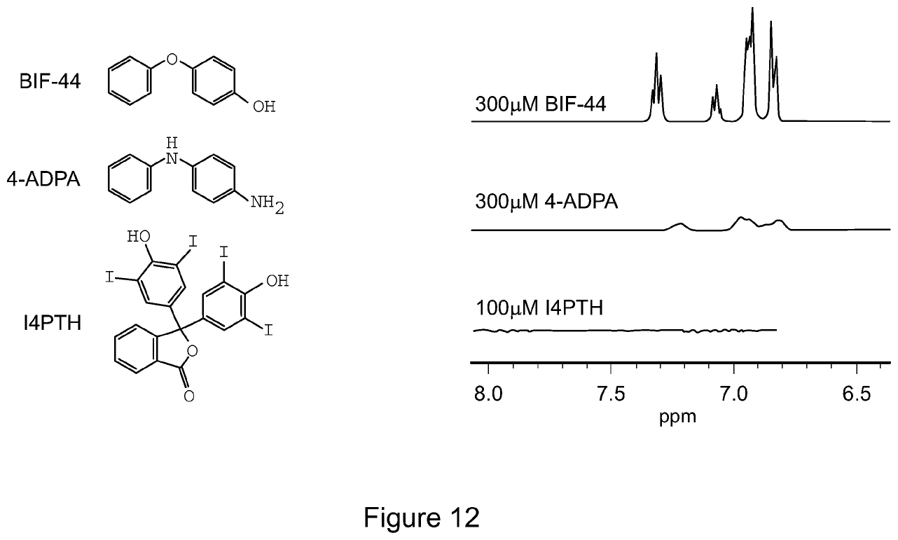

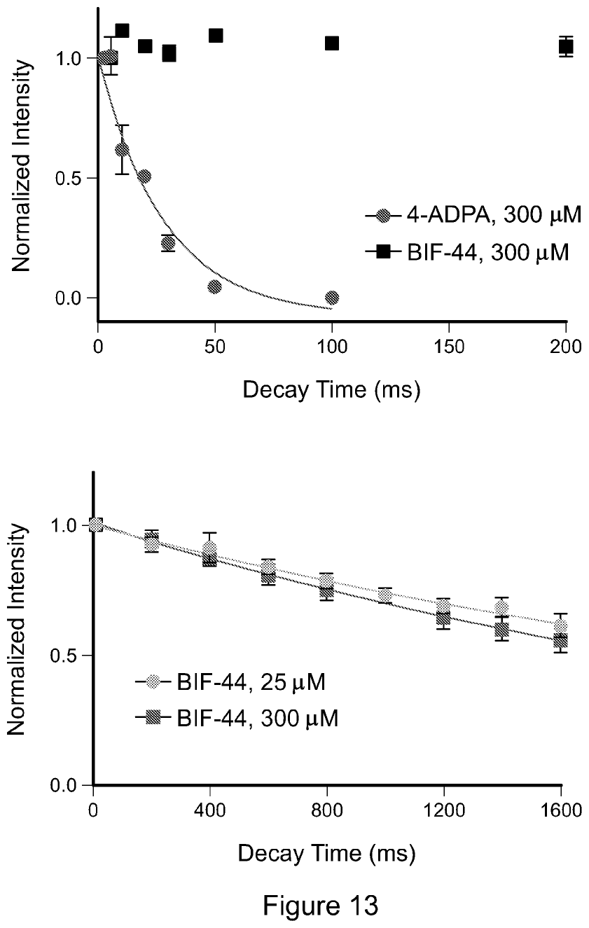

To detect line broadening, standard ¹H-NMR spectra were acquired. T2 decay curves were generated by measuring the CPMG NMR spectra of the molecules, performed as described above. The number of echo cycles corresponds to the decay time. The intensity of the aromatic peaks at the indicated decay times were measured and normalized to a maximum intensity of 1 at the 10 ms decay time. The curves were fitted to a one phase decay model using Prism software (Graphpad). Excitation sculpting was used for solvent suppression. Samples for both analyses were prepared in 20 mM potassium phosphate buffer, pH 6.2, 10% (v/v) D₂O. Results of these measurements are described in FIGS. 12-13.

### Example 15. Dynamic Light Scattering

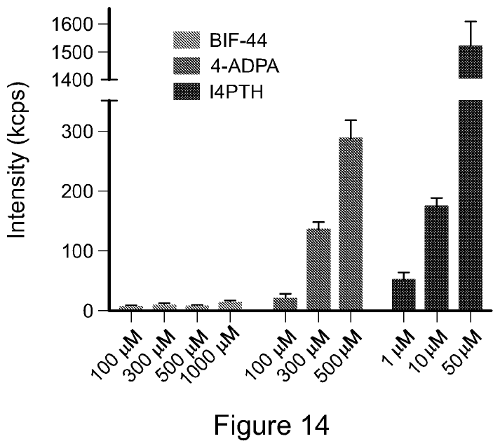

Samples were measured at room temperature on a DynaPro-99 instrument with a 90° detector angle using a 10 second acquisition time per measurement. Compounds were diluted from a 100 mM stock in 20 mM potassium phosphate buffer, pH 6.2 with 1% DMSO final concentration. Results of the dynamic light scattering measurements are shown in FIG. 14.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims. It should be appreciated by those persons having ordinary skill in the art(s) to which the present invention relates that any of the features described herein in respect of any particular embodiment of the present invention can be combined with one or more of any of the other features of any other embodiments of the present application described herein, with modifications as appropriate to ensure compatibility of the combinations. Such combinations are considered to be part of the present application.

1. A composition, comprising a compound of Formula I: 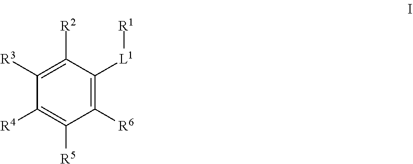
or a pharmaceutically acceptable salt thereof, and one or more pharmaceutically acceptable carriers, wherein:
L¹ is selected from the group consisting of a bond, C₁₋₃ alkylene, —O—, —O(C₁₋₃ alkylene)-, C₁₋₃ cyanoalkylene, —S—, —SO₂—, —S(C₁₋₃ alkylene)-, and —C(O)—;
R¹ is selected from the group consisting of halo, OH, C₁₋₃ alkyl, C₁₋₃ haloalkyl, NH₂, CN, phenyl, 5-6 membered heteroaryl, and 5-6 membered heterocycloalkyl, wherein the phenyl, 5-6 membered heteroaryl, and 5-6 membered heterocycloalkyl are each optionally substituted by 1, 2, or 3 independently selected R^{A} groups;
R² is selected from the group consisting of H, halo, OH, CN, C₁₋₃ alkyl, and C(O)OC₁₋₃ alkyl;
R³ is selected from the group consisting of H, halo, OH, NH₂, C(O)C₁₋₃ alkyl, and C(S)C₁₋₃ alkyl;
R⁴ is selected from the group consisting of H, halo, OH, NH₂, CN, C₁₋₃ alkyl, C₁₋₃ haloalkyl, and O(C₁₋₃ cyanoalkyl);
R⁵ is selected from the group consisting of H, halo, OH, NH₂, and C(O)C₁₋₃ alkyl;
R⁶ is selected from the group consisting of H, halo, OH, CN, C₁₋₃ alkyl, and C(O)OC₁₋₃ alkyl; and
each R^{A} is independently selected from the group consisting of OH, NH₂, CN, C₁₋₃ alkyl, C₁₋₃ hydroxyalkyl, C(O)OH, C(O)C₁₋₃ alkyl, and C(O)N(C₁₋₃ alkyl)₂, wherein the C₁₋₃ alkyl group is optionally substituted by NH₂.

2. The composition of claim 1, wherein L¹ is selected from the group consisting of a bond, —CH₂—, —O—, —OCH₂—, —CH(CN)—, —S—, —SO₂—, —SCH₂—, and —C(O)—.

3. The composition of claim 1, wherein L¹ is —O—, —CH₂—, or —OCH₂—.

4. The composition of claim 1, wherein R¹ is selected from the group consisting of Cl, CH₃, CF₃, NH₂, CN, phenyl, 5-6 membered heteroaryl, and 5-6 membered heterocycloalkyl, wherein the phenyl, 5-6 membered heteroaryl, and 5-6 membered heterocycloalkyl are each optionally substituted by 1 or 2 independently selected R^{A} groups.

5. The composition of claim 1, wherein R¹ is selected from the group consisting of Cl, CH₃, CF₃, NH₂, CN, phenyl, pyridyl, furanyl, thienyl, pyrrolyl, thiazolyl, oxazolyl, pyrazolyl, 1,2,4-thiadiazolyl, piperidinyl, morpholinyl, and 4,5-dihydrothiazolyl wherein the phenyl, pyridyl, furanyl, thienyl, pyrrolyl, thiazolyl, oxazolyl, pyrazolyl, 1,2,4-thiadiazolyl, piperidinyl, morpholinyl, and 4,5-dihydrothiazolyl are each optionally substituted by 1 or 2 independently selected R^{A} groups.

6. The composition of claim 1, wherein each R^{A} is independently selected from the group consisting of OH, NH₂, CN, CH₃, CH₂OH, CH₂CH₂NH₂, C(O)OH, C(O)CH₃, and C(O)N(CH₃)₂.

7. The composition of claim 1, wherein R¹ is phenyl which is optionally substituted by 1 or 2 independently selected R^{A} groups.

8. The composition of claim 1, wherein R¹ is phenyl, 4-hydroxyphenyl, 3-hydroxyphenyl, 4-aminophenyl, 4-carboxylphenyl, or 4-hydroxymethylphenyl.

9. The composition of claim 1, wherein R² is selected from the group consisting of H, Cl, CN, CH₃, and C(O)OCH₃.

10. The composition of claim 1, wherein R² is H or CH₃.

11. The composition of claim 1, wherein R³ is selected from the group consisting of H, F, C₁, NH₂, C(O)CH₃, and C(S)CH₃.

12. The composition of claim 1, wherein R³ is H.

13. The composition of claim 1, wherein R⁴ is selected from the group consisting of H, Cl, NH₂, CN, CH₃, CF₃, and OCH₃CN.

14. The composition of claim 1, wherein R⁴ is H or OH.

15. The composition of claim 1, wherein R⁵ is selected from the group consisting of H, F, Cl, NH₂, and C(O)CH₃.

16. The composition of claim 1, wherein R⁵ is H or NH₂.

17. The composition of claim 1, wherein R⁶ is selected from the group consisting of H, Cl, CN, CH₃, and C(O)OCH₃.

18. The composition of claim 1, wherein R⁶ is H.

19. The composition of claim 1, wherein the compound of Formula I is selected from the group consisting of: 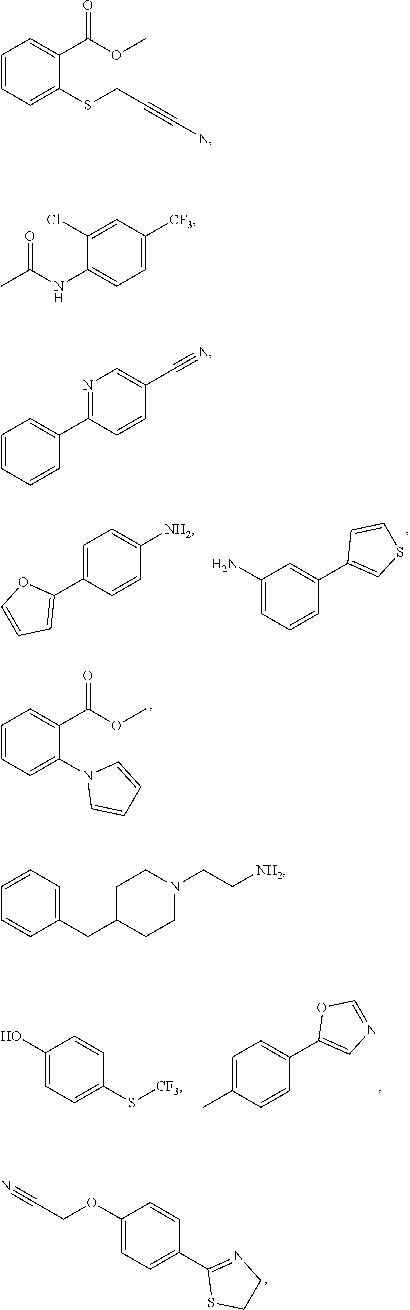 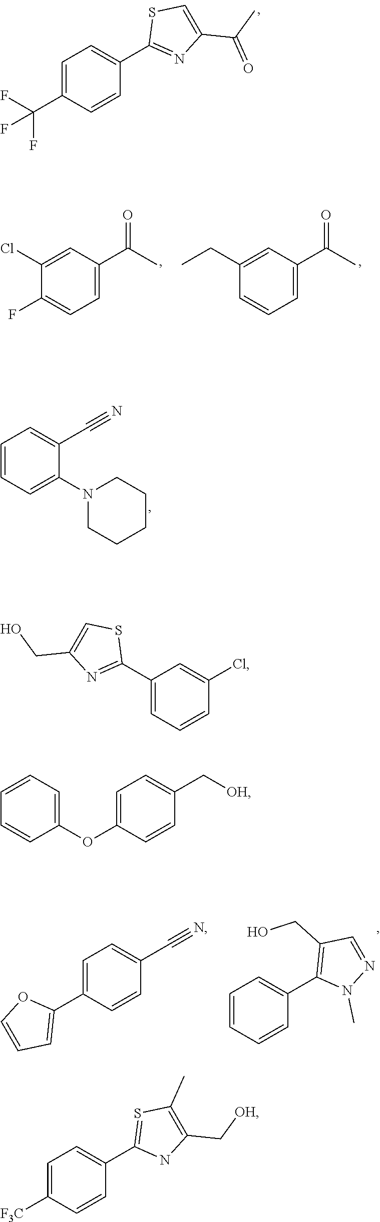 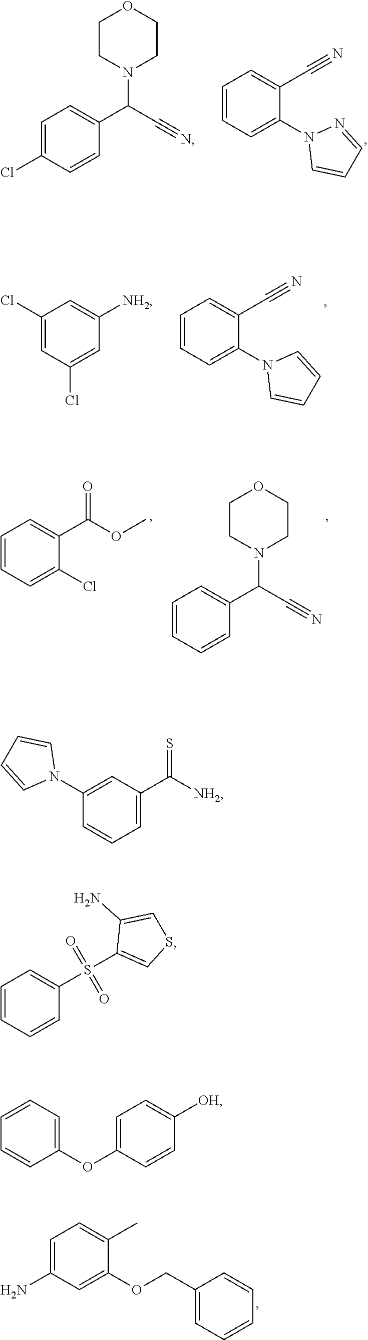 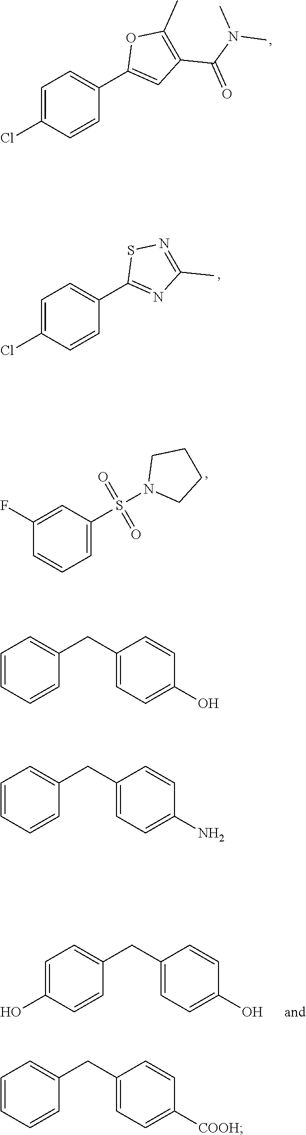
or a pharmaceutically acceptable salt thereof.

20. The composition of claim 1, wherein the compound of Formula I is: 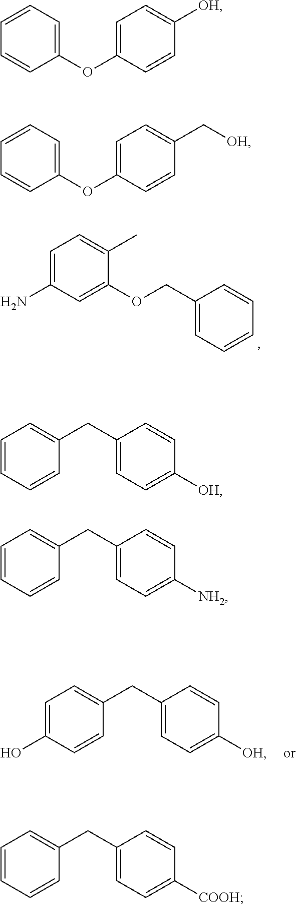
or a pharmaceutically acceptable salt thereof.

21. A composition, comprising a compound of Formula II: 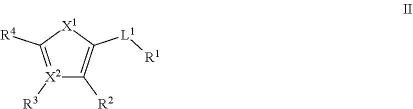
or a pharmaceutically acceptable salt thereof, and one or more pharmaceutically acceptable carriers, wherein:
X¹ is NH or S;
X² is C or N;
L¹ is selected from the group consisting of a bond, —C(O)—, —C(O)O—, and —SO₂—;
R¹ is selected from the group consisting of C₁₋₃ alkyl, NH₂, di(C₁₋₃ alkyl)amino, and a 5-6 membered heterocycloalkyl;
R² is selected from the group consisting of H, halo, C₁₋₃ alkyl, and C(O)OC₁₋₃ alkyl;
R³ is selected from the group consisting of H, C₁₋₃ alkyl, and 5-6 membered heteroaryl; or R³ is absent when X² is N; and
R⁴ is selected from the group consisting of H and C₁₋₃ alkyl.

22-31. (canceled)

32. A composition, comprising a compound of Formula III: 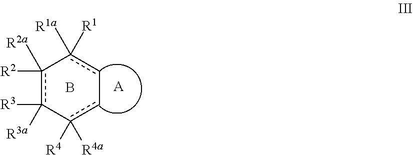
or a pharmaceutically acceptable salt thereof, and one or more pharmaceutically acceptable carriers, wherein: [symbol]
refers to a single bond or a double bond;
Ring A forms a fused ring with Ring B and Ring A is selected from the group consisting of a 5-6 membered cycloalkyl, a 5-6 membered heteroaryl, and a 5-6 membered heterocycloalkyl, wherein Ring A is optionally substituted by 1, 2, or 3 independently selected R^{A} groups;
R¹ is selected from the group consisting of H, C(O)OC₁₋₃ alkyl, OC(O)C₁₋₃ alkyl, C(S)NH₂, and ═N—OH;
R^{1a} is H; or R^{1a} is absent when the carbon atom to which R^{1a} is attached forms a double bond;
R² is selected from the group consisting of H and halo;
R^{2a} is H; or R^{2a} is absent when the carbon atom to which R^{2a} is attached forms a double bond;
R³ is selected from the group consisting of H, halo, C₁₋₃ alkyl, C₁₋₃ hydroxyalkyl, NHC(O)C₁₋₃ alkyl, and (C₁₋₃ alkylene)NHC₁₋₃ alkyl;
R^{3a} is C₁₋₃ alkyl; or R^{3a} is absent when the carbon atom to which R^{3a} is attached forms a double bond;
R⁴ is selected from the group consisting of H and C₁₋₃ alkyl;
R^{4a} is H; or R^{4a} is absent when the carbon atom to which R^{4a} is attached forms a double bond;
and
each R^{A} is independently selected from the group consisting of ═O, ═S, CN, C₁₋₃ alkyl, C₁₋₃ hydroxyalkyl, S(C₁₋₃ alkyl), and C(O)OH.

33-46. (canceled)

47. A composition, comprising a compound comprising a moiety of Formula IV: 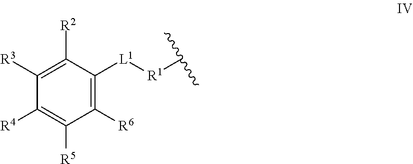
or a pharmaceutically acceptable salt thereof, and one or more pharmaceutically acceptable carriers, wherein:
L¹ is selected from the group consisting of a bond, C₁₋₃ alkylene, —O—, —O(C₁₋₃ alkylene)-, C₁₋₃ cyanoalkylene, —S—, —SO₂—, —S(C₁₋₃ alkylene)-, and —C(O)—;
R¹ is selected from the group consisting of phenylene, 5-6 membered heteroarylene, and 5-6 membered heterocycloalkylene, each of which is optionally substituted by 1, 2, or 3 independently selected R^{A} groups;
R² is selected from the group consisting of H, halo, OH, CN, C₁₋₃ alkyl, and C(O)OC₁₋₃ alkyl;
R³ is selected from the group consisting of H, halo, OH, NH₂, C(O)C₁₋₃ alkyl, and C(S)C₁₋₃ alkyl;
R⁴ is selected from the group consisting of H, halo, OH, NH₂, CN, C₁₋₃ alkyl, C₁₋₃ haloalkyl, and O(C₁₋₃ cyanoalkyl);
R⁵ is selected from the group consisting of H, halo, OH, NH₂, and C(O)C₁₋₃ alkyl;
R⁶ is selected from the group consisting of H, halo, OH, CN, C₁₋₃ alkyl, and C(O)OC₁₋₃ alkyl; and
each R^{A} is independently selected from the group consisting of OH, NH₂, CN, C₁₋₃ alkyl, C₁₋₃ hydroxyalkyl, C(O)OH, C(O)C₁₋₃ alkyl, and C(O)N(C₁₋₃ alkyl)₂, wherein the C₁₋₃ alkyl group is optionally substituted by NH₂.

48-55. (canceled)

56. A method of sensitizing and/or activating pro-apoptotic activity of BAX, comprising contacting a cell sample or tissue sample comprising BAX with a composition of claim 1.

57. A method of sensitizing and/or activating pro-apoptotic activity of BAX in a subject, comprising administering to the subject a composition of claim 1.

58. A method of treating cancer in a subject, comprising administering to the subject a therapeutically effective amount of a composition of claim 1.

59. The method of claim 58, wherein the cancer is selected from the group consisting of breast cancer, prostate cancer, lymphoma, skin cancer, pancreatic cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma.

60. The method of claim 58, wherein the cancer is leukemia.

61. The method of claim 60, wherein the leukemia is selected from the group consisting of acute lymphocytic leukemia, chronic lymphocytic leukemia, chronic granulocytic leukemia, acute granulocytic leukemia, acute myelogenous leukemia, chronic myelogenous leukemia, and hairy cell leukemia.

62. The method of claim 60, wherein the leukemia is selected from the group consisting of acute lymphoblastic leukemia, acute myelogenous leukemia, chronic lymphoblastic leukemia, and chronic myelogenous leukemia.

63. A method for identifying a compound which sensitizes or activates pro-apoptotic activity of a BAX polypeptide, the method comprising:
a) contacting a binding site of said BAX polypeptide comprising an amino acid sequence of SEQ ID NO:1 with a compound in vitro under conditions suitable for sensitizing or activating the pro-apoptotic activity of the BAX polypeptide; and
b) determining whether the compound binds to one or more amino acid residues selected from the group consisting of Ile80, Ala81, Ala82, Va183, Asp84, Thr85, Asp86, Ser87, Pro88, Val91, Phe116, Lys119, Leu120, Val121, Lys123, Ala124, Thr127, Leu132, and Ile136; wherein the binding site of the BAX polypeptide comprises the junction of the α3-α4 and α5-α6 hairpins of the BAX polypeptide.

64-68. (canceled)

69. The method of claim 56, wherein the composition sensitizes activation of the pro-apoptotic activity of the BAX polypeptide.

70. The method of claim 56, wherein the composition activates the pro-apoptotic activity of the BAX polypeptide.

71. The method of claim 56, wherein the method further comprises administration of an additional therapeutic agent which activates pro-apoptotic activity of BAX.

72. The method of claim 71, wherein the additional therapeutic agent is BIM SAHB_{A2}.


SEQUENCE LISTING (3 sequences)

SEQ ID NO: 1 - protein, 192 aa, Homo sapiens
MDGSGEQPRGGGPTSSEQIMKTGALLLQGFIQDRAGRMGGEAPELALDPVPQDASTKKLSECLKRIGDELDSNMELQRMIAAVDTDSPREVFFRVAADMFSDGNFNWGRVVALFYFASKLVLKALCTKVPELIRTIMGWTLDFLRERLLGWIQDQGGWDGLLSYFGTPTWQTVTIFVAGVLTASLTIWKKMG

SEQ ID NO: 2 - protein, 20 aa, Artificial Sequence
  Description of Artificial Sequence Synthetic peptide
EALKKALRRHRFLWQRRQRA

SEQ ID NO: 3 - protein, 20 aa, Artificial Sequence
  Description of Artificial Sequence Synthetic peptide
  MOD_RES (10)..(10): Any stapled amino acid
  MOD_RES (14)..(14): Any stapled amino acid
EIWIAQELRXIGDXFNAYYA